(19) United States
(12) Patent Application Publication
(10) Pub. No.: US 2025/0170074 A1
(43) Pub. Date: May 29, 2025

(54) PRECURSORS FOR THE CONTROLLED RELEASE OF A,B-UNSATURATED ALDEHYDES OR KETONES AND OTHER HIGH IMPACT FRAGRANCE SUBSTANCES

(71) Applicant: SYMRISE AG, Holzminden (DE)

(72) Inventors: Frank GERSTMANN, Frankfurt am Main (DE); Bernd HÖLSCHER, Halle (DE); Thorsten JUNGE, Holzminden (DE); Jörg EILERS, Holzminden (DE); Lucie KEPLER-BOZKURT, Holzminden (DE); Stephanie KORTE, Höxter (DE); Marc VOM ENDE, Berlin (DE); Franziska ELTERLEIN, Holzminden (DE); Philip KRAFT, Holzminden (DE)

(73) Assignee: SYMRISE AG, Holzminden (DE)

(21) Appl. No.: 18/681,244

(22) PCT Filed: May 4, 2023
(86) PCT No.: PCT/EP2023/061751

§ 371 Date: Feb. 5, 2024

(30) Foreign Application Priority Data
May 4, 2022 (WO) PCT/EP2022/062021

(51) Int. Cl. A61K 31/11; A61K 31/343; A61K 31/351; A61K 31/357; A61Q 13/00; C07C 323/12; C07C 323/13; C07C 323/22; C07D 307/00; C07D 309/04; C07D 319/06; C11D 3/34; C11D 3/50

(52) U.S. Cl. CPC A61K 31/11; A61K 31/343; A61K 31/351; A61K 31/357; A61Q 13/00; C07C 323/12; C07C 323/13; C07C 323/22; C07D 307/00; C07D 309/04; C07D 319/06; C11D 3/3427; C11D 3/50

(57) ABSTRACT

The present invention primarily relates to compounds of formula (I) as defined herein. It also relates to fragrance substance mixtures as defined herein and methods for producing the same and to perfumed products as defined herein and methods for producing the same. The present invention further relates to the use of a compound of formula (I) as defined herein as a precursor for the release of two or more different pleasantly smelling fragrance substances, to methods for the release of two or more different pleasantly smelling fragrance substances and to thiols as defined herein.

The present invention primarily relates to compounds of formula (I) as defined herein. It also relates to fragrance substance mixtures as defined herein and methods for producing the same and to perfumed products as defined herein and methods for producing the same. The present invention further relates to the use of a compound of formula (I) as defined herein as a precursor for the release of two or more different pleasantly smelling fragrance substances, to methods for the release of two or more different pleasantly smelling fragrance substances and to thiols as defined herein.

Further aspects and preferred embodiments of the present invention are apparent from the following description, the attached examples and, in particular, the attached patent claims.

On the part of the perfume industry, there is a particular interest in a controlled and long-lasting release of certain fragrances. This applies in particular to fragrances with a high volatility or low substantivity. The washing of textiles, for example, is an area where there is a great desire to be able to perceive the pleasant odours of the fragrance substances or perfume oils used in detergents or fabric softeners on the laundry for a long period of time after washing and drying of the textiles.

Precursors are known in the perfume industry and are formed, for example, from α,β-unsaturated carbonyl compounds and thiols by way of Michael additions. They usually release a pleasantly smelling α,β-unsaturated carbonyl compound, such as an α,β-unsaturated aldehyde or ketone. This release takes place in a controlled manner and thus enables a sensorial perception of highly volatile fragrance substances that would not be achievable if the aldehyde or ketone compounds were used directly in a perfume oil.

There is a great demand in the perfume industry for precursors that are capable of releasing several fragrance substances simultaneously or over time. This would allow an expansion of the perfumery palette. There is also a particularly high demand for fragrance substances that have a strong olfactory effect even at low dosages (i.e. a low odorant threshold), so-called “high impact” fragrance substances.

The known precursors are limited to the release of fragrant α,β-unsaturated carbonyl compounds. Due to the unpleasant odour impression of common, especially short-chain, thiol compounds, the prior art teaches the use of thiol compounds that are as odourless as possible for the formation of precursors. In patent application WO 2004/105713 A1, for example, 3-mercaptopropyltriethoxysilanes and 3-mercaptopropyltrimethoxysilanes are reacted with α,β-unsaturated carbonyl compounds to form precursors. Only the released α,β-unsaturated carbonyl compounds serve as fragrance substances in the teaching described therein.

In patent application WO 2015/032885 A1, short-chain thiol compounds are used as thiol compounds to form the precursors. Also in this publication, only the α,β-unsaturated carbonyl compounds released from the precursors serve as fragrance substances. It is described that the unpleasant odour of the short-chain thiol compounds, which are also released from the precursors, surprisingly does not affect the positive olfactory effect of the α,β-unsaturated carbonyl compounds too negatively, so that overall, unexpectedly, the positive olfactory impression of the released α,β-unsaturated carbonyl compounds dominates.

The primary task was to provide improved precursors that meet the above requirements, i.e. are capable of releasing pleasantly smelling, high impact fragrances in a controlled manner and over a long period of time. This release should preferably be possible from surfaces treated with the precursors, especially from textiles.

Surprisingly, this task is solved by a compound of formula (I)

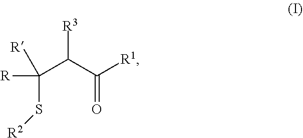

(i) wherein R is selected from the group consisting of hydrogen and C₁ to C₁₀, preferably C₁ to C₉, more preferably C₁ to C₈, more preferably C₁ to C₇, more preferably C₁ to C₉, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
   wherein R′ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
   wherein R¹ is selected from the group consisting of hydrogen and C₁ to C₁₀, preferably C₁ to C₉, more preferably C₁ to C₈, more preferably C₁ to C₇, more preferably C₁ to C₉, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
   wherein R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, preferably additionally comprising one, two or more functional groups selected from the group consisting of ether, alcohol, and ketone, more preferably additionally comprising one ether group or two ether groups or one alcohol group or one ketone group, and
   wherein R³ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another),
or
(ii) wherein R¹, R² and R³ are independently selected from the groups as defined in (i), respectively, and R and R¹ are bonded together to form a saturated or unsaturated ring system comprising 5 to 10 carbon atoms, preferably 6 to 8 carbon atoms, most preferably 6 carbon atoms, preferably wherein the ring system additionally carries one, two, three, four or more C₁ to C₅ linear, branched or cyclic alkyl, alkenyl or alkadienyl substituents (and wherein the substituents can be the same or different from one another),
or
(iii) wherein R¹, R¹ and R² are independently selected from the groups as defined in (i), respectively, and R and R³ are bonded together to form a saturated or unsaturated, mono or bicyclic, ring system comprising 5 to 10 carbon atoms and optionally 1 or 2 ether functional groups, preferably comprising 6 to 8 carbon atoms and optionally 1 ether functional group, most preferably comprising 6 carbon atoms, preferably wherein the ring system additionally carries one, two, three, four or more C₁ to C₈ linear, branched or cyclic alkyl, alkenyl or alkadienyl substituents, preferably one, two or more methyl groups.

The ring system as defined in (ii) above includes the carbon atoms to which R and R¹ are bonded and the ring system as defined in (iii) above includes the carbon atoms to which R and R³ are bonded.

The compound of formula (I) (i.e. the precursor) may be in the form of a pure diastereomer or stereoisomer or in the form of any mixture of said isomers.

The compounds of formula (I) as defined herein have not been previously known in the perfume industry. The suitability of the compounds of formula (I) as precursors for the release of fragrance substances was therefore unexpected. In the course of the studies underlying this application, and contrary to the teaching in the prior art with respect to the negative olfactory influence of the released thiol compound, it was surprisingly found that the compounds of formula (I) according to the present invention release both a pleasantly smelling (high impact) α,β-unsaturated aldehyde or ketone and a pleasantly smelling (high impact) thiol compound. Said release of the two (or more) different fragrance substances, and thus their sensorial perception, advantageously occurs in a controlled way, preferably at different points in time, which was particularly surprising. The sensory odour impression of the released thiol compound (e.g. sulphurous-fruity notes), for instance, is very intensely perceptible on damp laundry, especially directly after washing, while the released α,β-unsaturated aldehyde or ketone provides a long-lasting, fresh odour impression on dried laundry (e.g. fresh, green and/or flowery notes).

Further advantages of the present invention are:
Due to the controlled release of two different pleasantly smelling, high impact fragrance substances with low odorant thresholds (α,β-unsaturated aldehyde or ketone compound, thiol compound), a distinct odour perception occurs when even small amounts of compounds of formula (I) according to the present invention are used.
A higher atomic economy is achieved compared to the precursors of the prior art, since, unexpectedly, both compounds released by the compounds of formula (I) according to the present invention (α,β-unsaturated aldehyde or ketone compound, thiol compound) contribute to a pleasant odour profile.
A positive effect of the compounds of formula (I) according to the present invention on the odour impression is surprisingly achieved both on wet laundry (thiol compound) as well as on dried laundry (α,β-unsaturated aldehyde or ketone compound), as the different fragrance substances are preferably released from the compounds of formula (I) according to the present invention and/or sensorially perceived at different points in time.
The thiol compound released by the compounds of formula (I) according to the present invention not only improves the substantivity and/or deposition of the compounds of formula (I) according to the present invention on e.g. textiles, hair or skin, but it also serves as a pleasantly smelling fragrance substance itself.

According to a preferred embodiment of the compound of formula (I) according to the invention,
R is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three, four or more C₁ to C₄ linear, cyclic or branched alkyl, alkenyl or alkadienyl substituents (and wherein the substituents can be the same or different from one another), and
R′ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three, four or more C₁ to C₄ linear, cyclic or branched alkyl, alkenyl or alkadienyl substituents (and wherein the substituents can be the same or different from one another), and
R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three, four or more C₁ to C₄ linear, cyclic or branched alkyl, alkenyl or alkadienyl substituents (and wherein the substituents can be the same or different from one another), and
R² is selected from the group consisting of C₆ to C₁₂, more preferably C₇ to C₁₂, most preferably C₈ to C₁₂, linear, cyclic or branched alkyl residues, preferably additionally comprising one, two or more functional groups selected from the group consisting of ether, alcohol, and ketone, more preferably additionally comprising one ether group or two ether groups or one alcohol group or one ketone group, and
R³ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three, four or more C₁ to C₄ linear, cyclic or branched alkyl, alkenyl or alkadienyl substituents (and wherein the substituents can be the same or different from one another).

According to another preferred embodiment of the compound of formula (I) according to the invention, R is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and
R′ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and
R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and
R² is selected from the group consisting of C₆ to C₁₂, more preferably C₇ to C₁₂, most preferably C₈ to C₁₂, linear, cyclic or branched alkyl residues, preferably additionally comprising one, two or more functional groups selected from the group consisting of ether, alcohol, and ketone, more preferably additionally comprising one ether group or two ether groups or one alcohol group or ketone group, and
R³ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another).

According to another preferred embodiment of the compound of formula (I) according to the invention, R is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and
R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and

R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl or alkenyl residues, preferably wherein the residues additionally carry one, two, three or more substituents selected from the group consisting of methyl and ethenyl (and wherein the substituents can be the same or different from one another), and
R² is selected from the group consisting of C₆ to C₁₂, more preferably C₇ to C₁₂, most preferably C₈ to C₁₂, linear, cyclic or branched alkyl residues, preferably additionally comprising one, two or more functional groups selected from the group consisting of ether, alcohol, and ketone, more preferably additionally comprising one ether group or two ether groups or one alcohol group or one ketone group, and
R³ is hydrogen.

Residue R² of the compounds of formula (I) according to the invention does not comprise any Si atoms.

According to another preferred embodiment of the compound of formula (I) according to the invention, R² is not an n-butyl, n-pentyl, or n-hexyl (or 2-propyl) residue.

According to another preferred embodiment of the compound of formula (I) according to the invention, R² is not a residue of the following formula (4-hydroxybutyl):

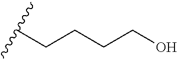 [SMILES: *CCCCO]

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one, two or more ether functional groups.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one ether functional group.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising two ether functional groups.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one, two or more alcohol functional groups.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one alcohol functional group.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising two alcohol functional groups.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one, two or more ketone functional groups.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising one ketone functional group.

According to an alternative preferred embodiment of the compound of formula (I) according to the invention, R² is selected from the group consisting of C₄ to C₁₅, preferably C₄ to C₁₂, more preferably C₅ to C₁₂, more preferably C₆ to C₁₂, more preferably C₇ to C₁₂, most preferably C₈ to C₁₂, linear, cyclic or branched alkyl residues, additionally comprising two ketone functional groups.

According to a preferred embodiment of the compound of formula (I) according to the invention, R is selected from the group consisting of

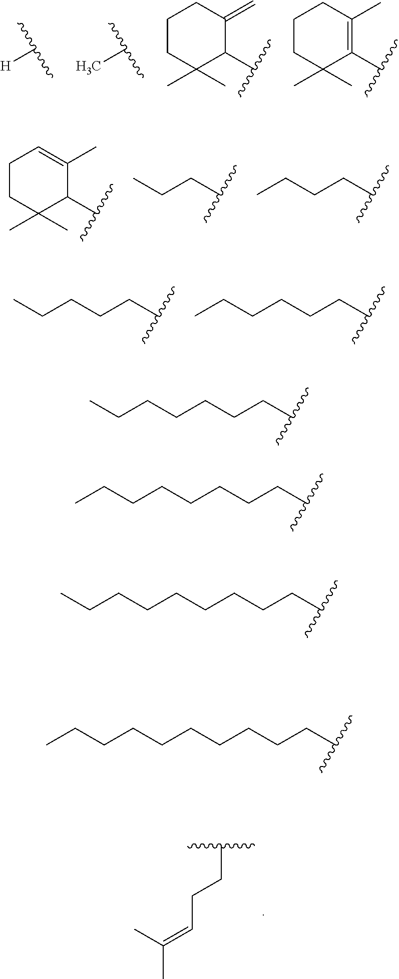

According to another preferred embodiment of the compound of formula (I) according to the invention, R′ is selected from the group consisting of

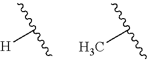

According to another preferred embodiment of the compound of formula (I) according to the invention, R¹ is selected from the group consisting of

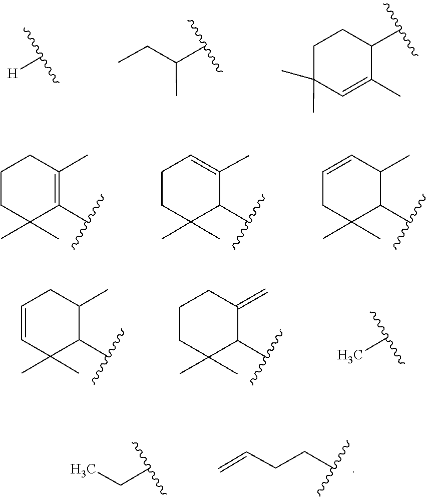

According to another preferred embodiment of the compound of formula (I) according to invention, R² is selected from the group consisting of

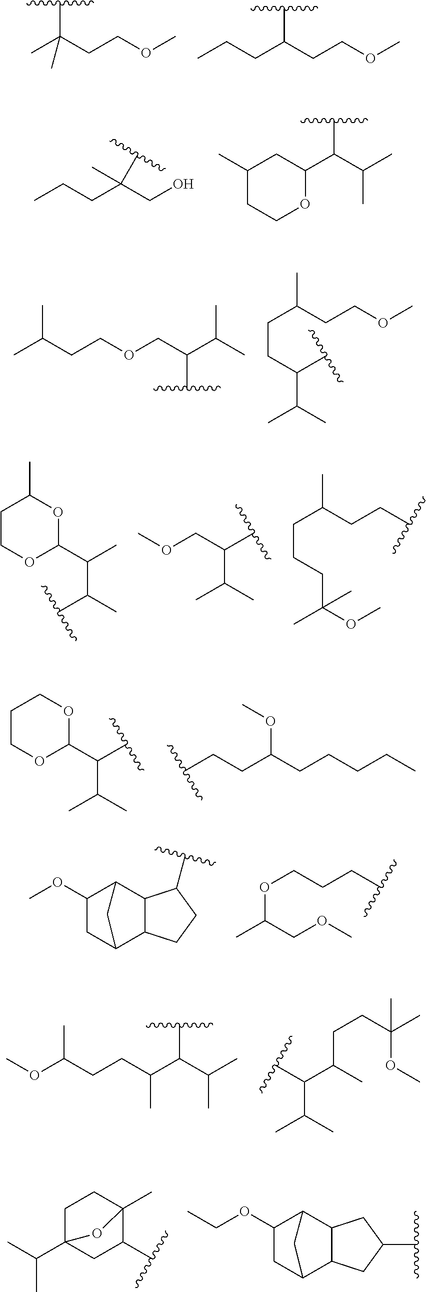

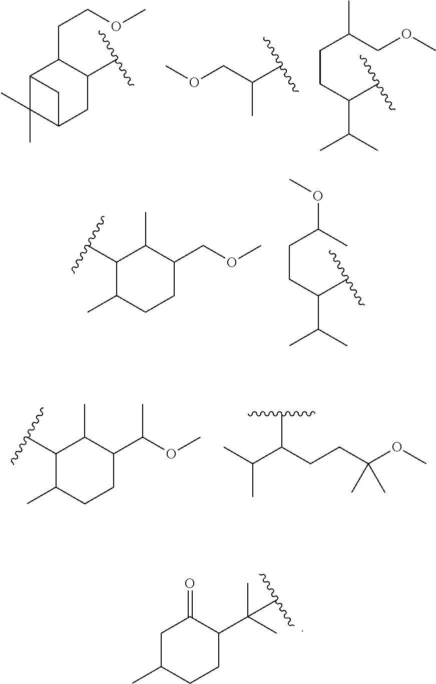

According to another preferred embodiment of the compound of formula (I) according to invention, R² is selected from the group consisting of

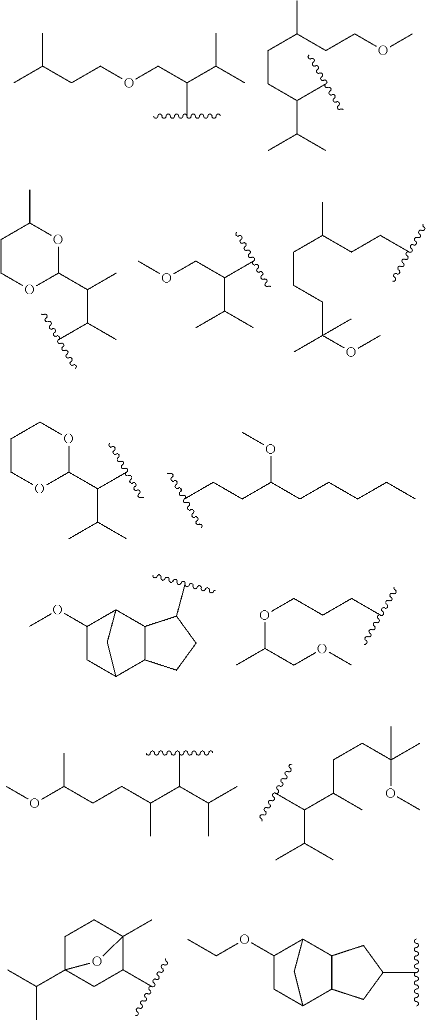

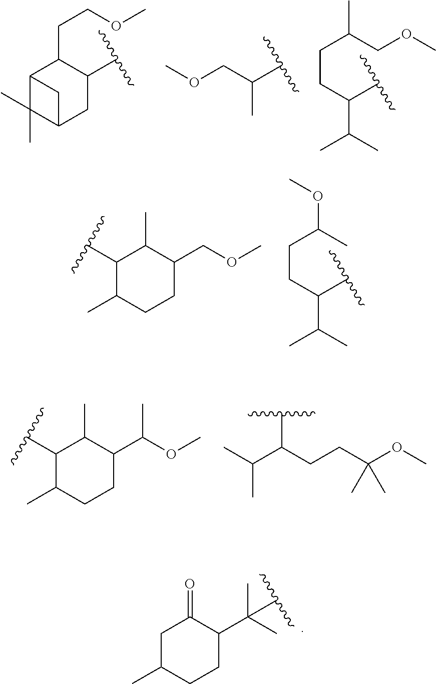

According to another preferred embodiment of the compound of formula (I) according to the invention, R is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and

R′ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and R² is selected from the group consisting of

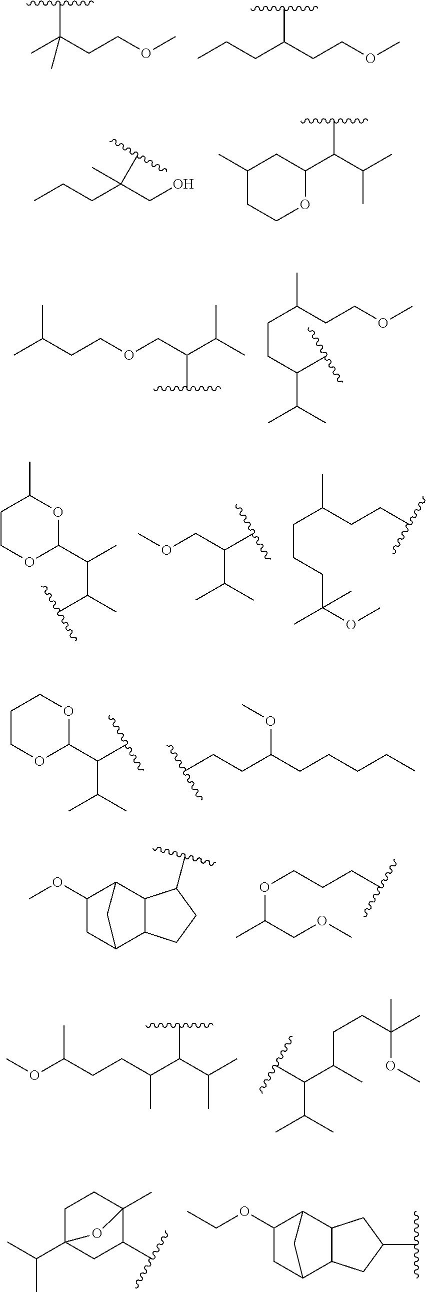

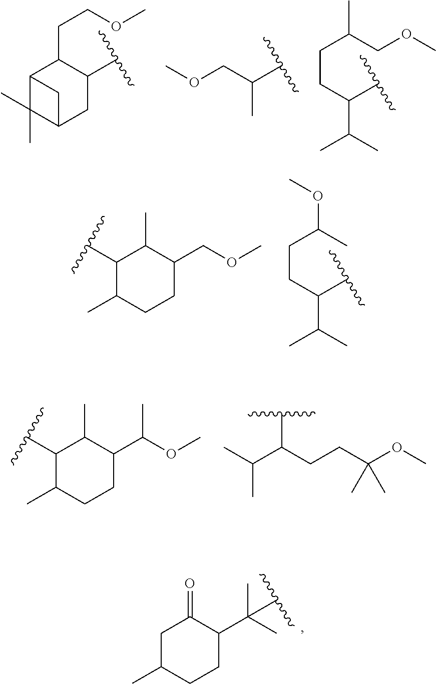

and
R³ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another).

According to a particularly preferred embodiment of the compound of formula (I) according to the invention,
R is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
R′ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
R¹ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another), and
R² is selected from the group consisting of

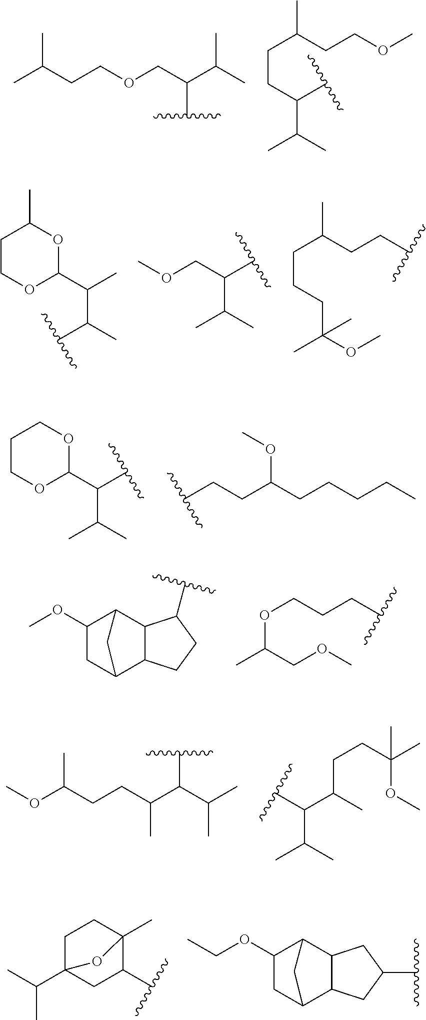

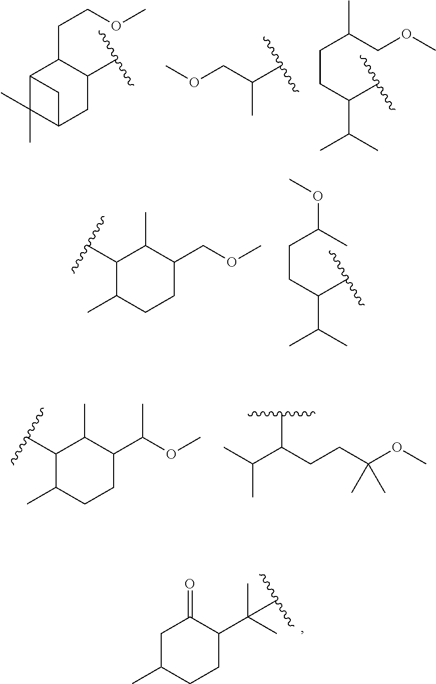

and
R³ is selected from the group consisting of hydrogen and C₁ to C₁₀, preferably C₁ to C₉, more preferably C₁ to C₈, more preferably C₁ to C₇, more preferably C₁ to C₆, most preferably C₁ to C₅, linear or cyclic alkyl, alkenyl or alkadienyl residues, preferably wherein the residues additionally carry one, two, three, four or more C₁ to C₄ linear, cyclic or branched alkyl, alkenyl or alkadienyl substituents (and wherein the substituents can be the same or different from one another).

According to another particularly preferred embodiment of the compound of formula (I) according to the invention, residue

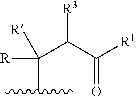

of the compound of formula (I) is selected from the group consisting of

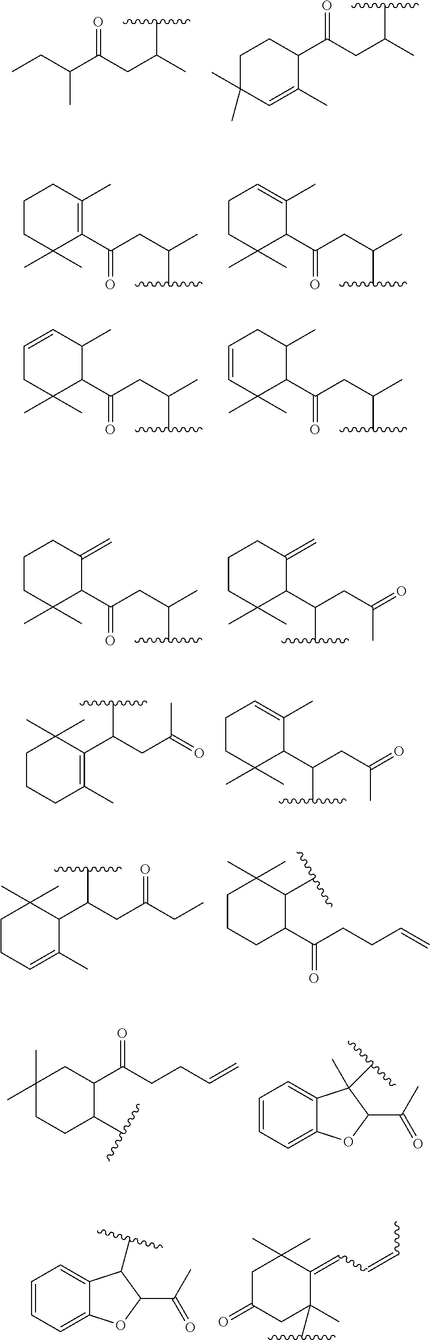

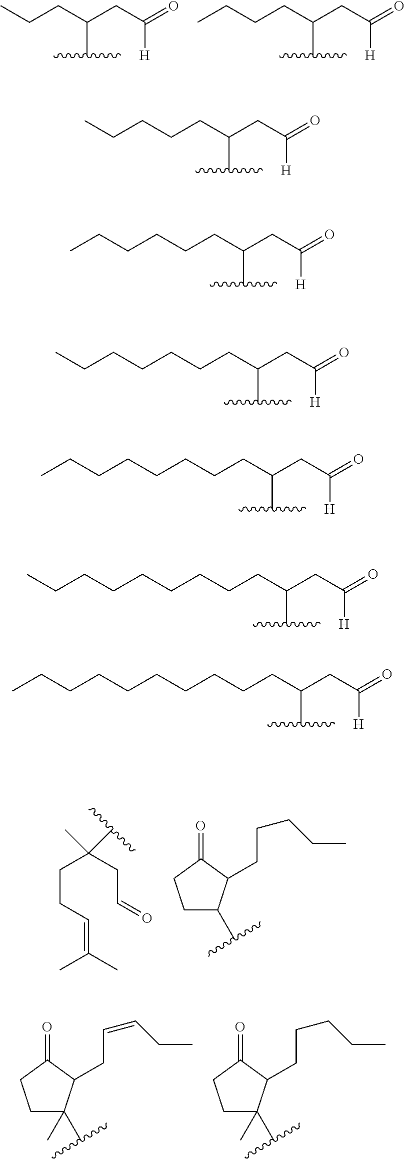

and/or, preferably and, residue R² of the compound of formula (I) is selected from the group consisting of

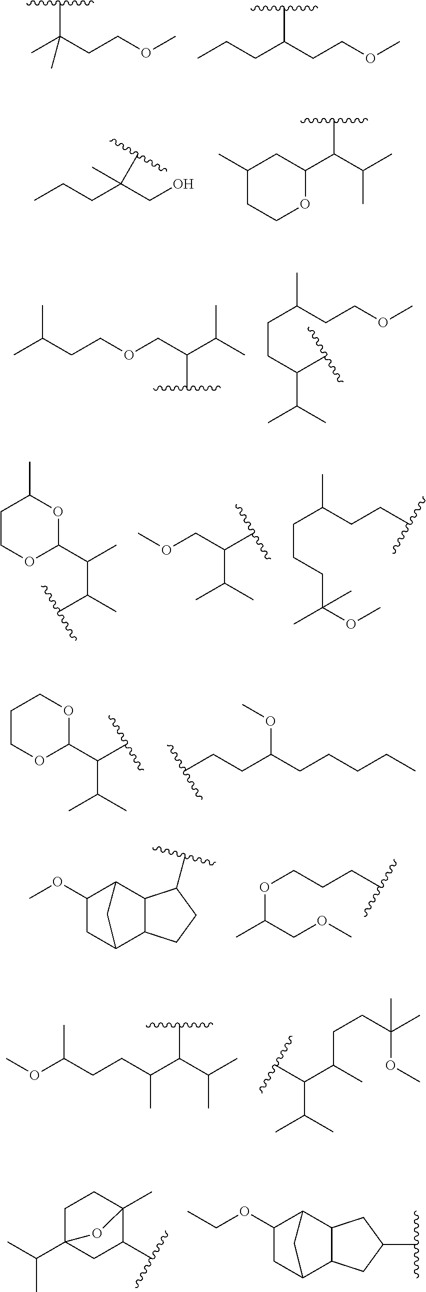

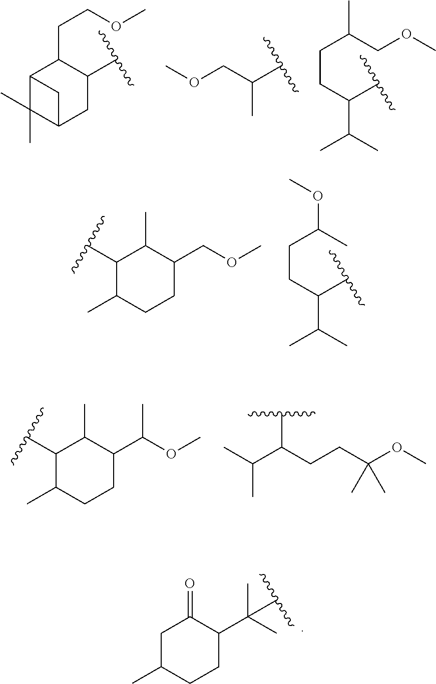

According to a preferred embodiment, the compound of formula (I) according to the invention is obtained or obtainable by a [1,4]-addition reaction between:
an α,β-unsaturated aldehyde or ketone (Michael acceptor, electrophile) selected from the group consisting of (group of compounds as shown below in the column entitled “Structure” of Table 1)

TABLE 1

| CAS | | |
|---|---|---|
| Number(s) | Name | Structure |
| 102322-83-8 | Filbertone | 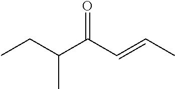 |
| 39872-57-6 | Isodamascone | 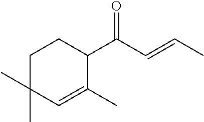 |
| 23726-91-2 | beta-Damascone | 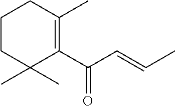 |
| 24720-09-0 | alpha-Damascone | 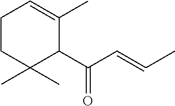 |
| 57378-68-4 | delta-Damascone | 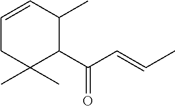 |
| 57350-34-2 | epsilon- Damascone | 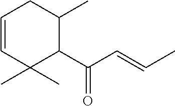 |
| 35076-56-3 | gamma- Damascone | 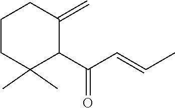 |
| 79-76-5 | gamma-Ionone | 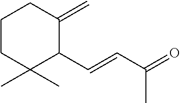 |
| 79-77-6 | beta-Ionone | 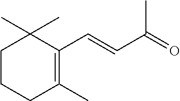 |
| 127-41-3 | alpha-Ionone | 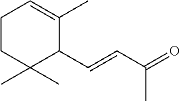 |
| 127-42-4 | Methyliridone | 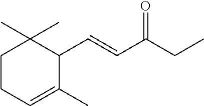 |
| 56973-84-3 | beta-Dynascone | 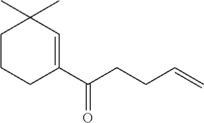 |
| 56973-85-4 | alpha-Dynascone | 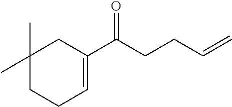 |
| 23911-56-0 | Nerolione | 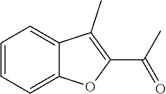 |
| 1646-26-0 | Coumarone | 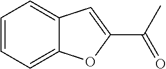 |
| 13215-88-8, 163440-97-9 (mixture of isomers) | Tabanone | 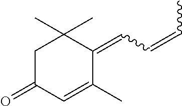 |
| 505-57-7 | 2-Hexenal | 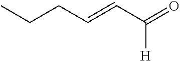 |
| 2463-63-0 | 2-Heptenal | 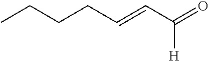 |
| 2363-89-5 | 2-Octenal | 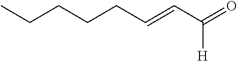 |
| 2463-53-8 | 2-Nonenal | 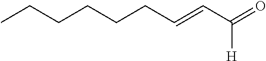 |
| 3913-71-1 | 2-Decenal | 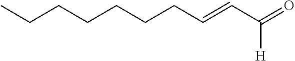 |
| 2463-77-6 | 2-Undecenal | 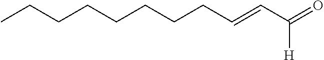 |
| 4826-62-4 | 2-Dodecenal | 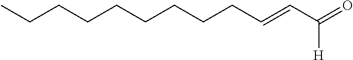 |
| 7774-82-5 | 2-Tridecenal | 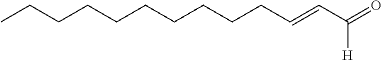 |
| 106-26-3 | Neral | 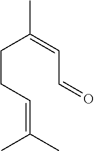 |
| 141-27-5 | Geranial | 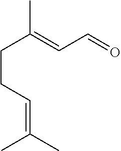 |
| 5392-40-5 (mixture of isomers) | Citral | 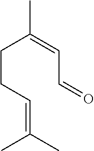 |
| | | 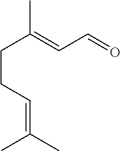 |
| | 2- Pentylcyclopent- 2-en-1-one | 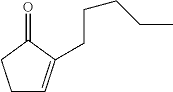 |
| | (Z)-3-Methyl-2- (pent-2-en-1- yl)cyclopent-2- en-1-one | 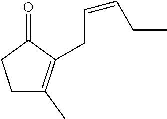 |
| | 3-Methyl-2- pentylcyclopent- 2-en-1-one | 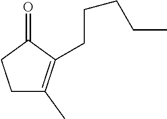 |

and
a thiol (Michael donor, nucleophile) selected from the group consisting of (group of compounds as shown below in the column entitled “Structure” of Table 2)

TABLE 2

| Chemical Name | Odour Description | Structure |
|---|---|---|
| 4-Methoxy-2-methyl-butane-2- thiol | — | 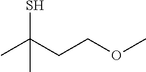 |
| 1-Methoxyhexane-3-thiol | — | 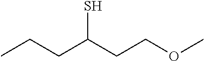 |
| 2-Methyl-2-sulfanyl-pentan-1- ol | Grapefruit, cassis, onion | 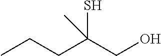 |
| 2-Methyl-1-(4- methyltetrahydropyran-2- yl)propane-1-thiol | Very powerful, more fruity, better quality, exotic fruit, guava | 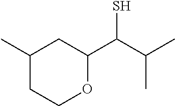 |
| 1-Isopentyloxy-3-methyl- butane-2-thiol | breath of smoker | 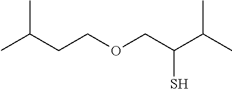 |
| 8-Methoxy-2,6-dimethyl- octane-3-thiol | Sulphur, grapefruit- sulphuric side, more fruity, cassis, plastic, green, rose oxide, green pepper | 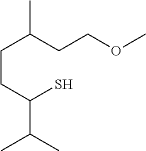 |
| 3-(4-Methyl-1,3-dioxan-2- yl)butane-2-thiol | Liver sausage, burnt plastic, buchu (Asian chives) note, cassis, gasoline | 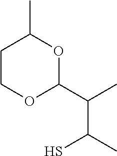 |
| 1-Methoxy-3-methyl-butane-2- thiol | Strong, more fruity, juicy, natural, cat grass, meat broth (“Fleischbrühe”), onion | 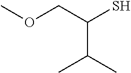 |
| 7-Methoxy-3,7-dimethyl- octane-1-thiol | Cheesy, camembert, cheese in the fridge | 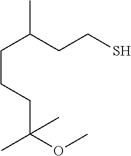 |
| 1-(1,3-Dioxan-2-yl)-2-methyl- propane-1-thiol | Fresh, strong, hot rubber, petrol | 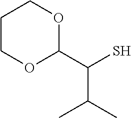 |
| 3-Methoxyoctane-1-thiol | Rose oxide, violet | 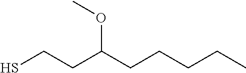 |
| Methoxytricyclo[5.2.1.0^{2,6}] decane-3-thiol | Blackcurrant, catnip, natural, fruity, zestral aspect, anisic | 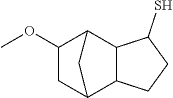 |
| 3-(2-Methoxy-1-methyl- ethoxy)propane-1-thiol | Minty green, catnip grass, mushroom, soup, leek | 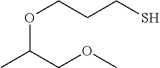 |
| 7-Methoxy-2,4-dimethyl- octane-3-thiol | Sulphur and something well known behind, agrumen, fruity, grapefruit but with natural aspect, odour direction: grapfruit natural | 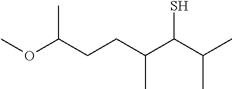 |
| 7-Methoxy-2,4,7-trimethyl- octane-3-thiol | Peach, cassis, meat, sulphur | 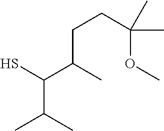 |
| 4-Isopropyl-1-methyl-7- oxabicyclo[2.2.1]heptane-2- thiol | — | 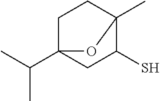 |
| 8-Ethoxy- tricyclo[5.2.1.0^{2,6}]decane-4- thiol | Cassis, sulphur | 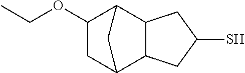 |
| 3-Ethyl-2-(2-methoxyethyl)- 4,4-dimethyl-cyclohexanethiol | Grapefruit, pine, meat, natural | 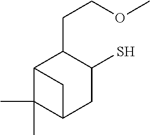 |
| 1-Methoxypropane-2-thiol | Mango, meat | 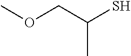 |
| 7-Methoxy-2,6-dimethyl- heptane-3-thiol | Sulphur, durian, jackfruit, artichoke | 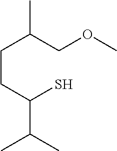 |
| 6-(Methoxymethyl)-2,3- dimethyl-cyclohexanethiol | Cassis, blackcurrant | 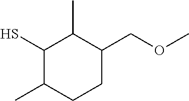 |
| 6-Methoxy-2-methyl-heptane- 3-thiol | Sulphur | 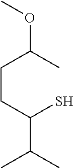 |
| 3-(1-Methoxyethyl)-2,6- dimethyl-cyclohexanethiol | Sulphur | 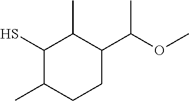 |
| 6-Methoxy-2,6-dimethyl- heptane-3-thiol | Grapefruit, fruity, apricot, peach | 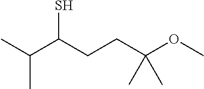 |
| 2-(2-Mercaptopropan-2-yl)-5- methylcyclohexan-1-one | Buchu | 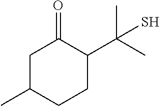 |

According to a preferred embodiment, Citral—i.e. a mixture of Neral and Geranial as shown above in Table 1—is used as (a mixture of) α,β-unsaturated aldehyde(s) for the formation of a compound of formula (I).

The compounds of formula (I) according to the invention

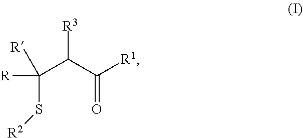

release a (pleasantly smelling, high impact) thiol, which is preferably selected from the group consisting of compounds as shown above in the column entitled “Structure” of Table 2, and a (pleasantly smelling, high impact) α,β-unsaturated aldehyde or ketone, which is preferably selected from the group consisting of compounds as shown above in the column entitled “Structure” of Table 1, via the following decomposition reaction:

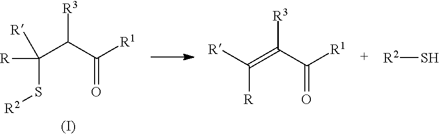

Preferably, said release is triggered by certain pH values and/or temperatures, UV light, water, and/or oxygen.

According to a particularly preferred embodiment, the compound of formula (I) according to the invention is selected from the group consisting of (group of compounds as shown below in the column entitled “Structure” of Table 3):

TABLE 3

| Chemical name | Structure |
|---|---|
| 3-[2-Methyl-1-(4- methyltetrahydropyran-2- yl)propyl]sulfanyl-1-(2,6,6- trimethylcyclohex-3-en-1-yl)butan-1- one | 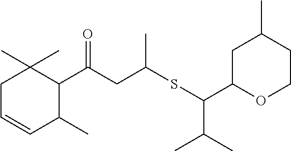 [SMILES: CC1CCOC(C(SC(C)CC(=O)C2C(C)C=CCC2(C)C)C(C)C)C1] |
| 3-[2-Methyl-1-(4- methyltetrahydropyran-2- yl)propyl]sulfanyldecanal | 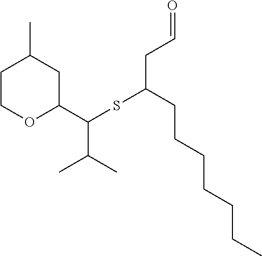 [SMILES: CCCCCCCC(CC=O)SC(C(C)C)C1CC(C)CCO1] |
| 2-[1-(Methoxymethyl)-2-methyl- propyl]sulfanyl-5-methyl-heptan-4-one | 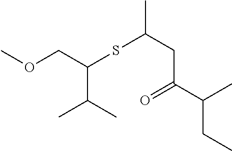 [SMILES: CCC(C)C(=O)CC(C)SC(COC)C(C)C] |
| 2-(3-Methoxyoctylsulfanyl)-5-methyl- heptan-4-one | 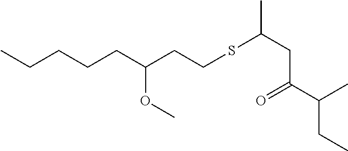 [SMILES: CCCCCC(CCSC(C)CC(=O)C(C)CC)OC] |
| 2-[(8-Ethoxy-4- tricyclo[5.2.1.02,6]decanyl)sulfanyl]-5- methyl-heptan-4-one | 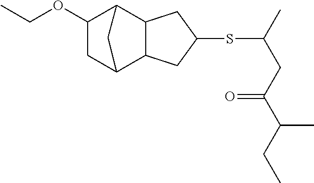 [SMILES: CCOC1CC2CC1C1CC(SC(C)CC(=O)C(C)CC)CC21] |
| 2-(2-Methoxy-1-methyl-ethyl)sulfanyl- 5-methyl-heptan-4-one | 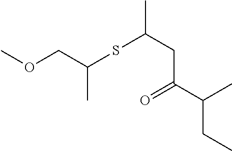 |
| 3-(3-Methoxy-1,1-dimethyl- propyl)sulfanyl-1-(2,6,6- trimethylcyclohex-3-en-1-yl)butan-1- one | 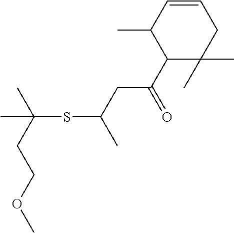 [SMILES: COCCC(C)(C)SC(C)CC(=O)C1C(C)C=CCC1(C)C] |
| 3-[1-(2-Methoxyethyl)butylsulfanyl]-1- (2,6,6-trimethylcyclohex-3-en-1- yl)butan-1-one | 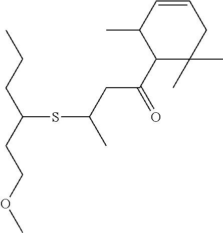 [SMILES: CCCC(CCOC)SC(C)CC(=O)C1C(C)C=CCC1(C)C] |
| 3-[1-(Hydroxymethyl)-1-methyl- butyl]sulfanyl-1-(2,6,6- trimethylcyclohex-3-en-1-yl)butan-1- one | 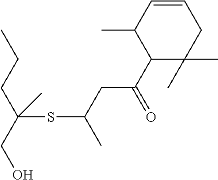 [SMILES: CCCC(C)(CO)SC(C)CC(=O)C1C(C)C=CCC1(C)C] |
| 3-(1-Isopropyl-6-methoxy-4-methyl- hexyl)sulfanyl-1-(2,6,6- trimethylcyclohex-3-en-1-yl)butan-1- one | 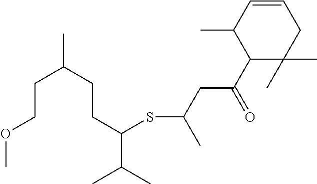 [SMILES: COCCC(C)CCC(SC(C)CC(=O)C1C(C)C=CCC1(C)C)C(C)C] |
| 3-[1-(Methoxymethyl)-2-methyl- propyl]sulfanyl-1-(2,6,6- trimethylcyclohex-3-en-1-yl)butan-1- one | 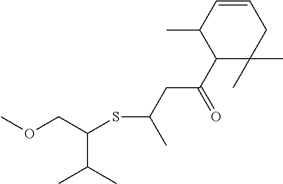 |
| 3-(3-Methoxyoctylsulfanyl)-1-(2,6,6- trimethylcyclohex-3-en-1-yl)butan-1- one | 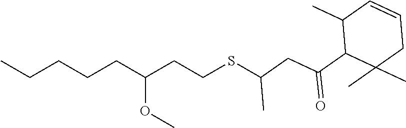 [SMILES: CCCCCC(CCSC(C)CC(=O)C1C(C)C=CCC1(C)C)OC] |
| 3-[(8-Ethoxy-4- tricyclo[5.2.1.02,6]decanyl)sulfanyl]-1- (2,6,6-trimethylcyclohex-3-en-1- yl)butan-1-one | 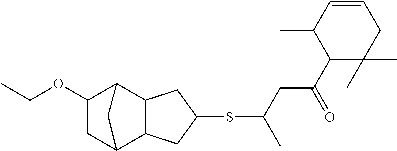 [SMILES: CCOC1CC2CC1C1CC(SC(C)CC(=O)C3C(C)C=CCC3(C)C)CC21] |
| 3-(2-Methoxy-1-methyl-ethyl)sulfanyl- 1-(2,6,6-trimethylcyclohex-3-en-1- yl)butan-1-one | 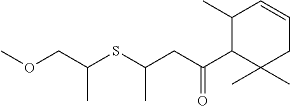 |
| 3-(1-Isopropyl-4-methoxy-4-methyl- pentyl)sulfanyl-1-(2,6,6- trimethylcyclohex-3-en-1-yl)butan-1- one | 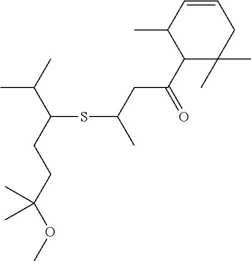 [SMILES: COC(C)(C)CCC(SC(C)CC(=O)C1C(C)C=CCC1(C)C)C(C)C] |
| 3-[1-(Methoxymethyl)-2-methyl- propyl]sulfanyl-1-(2,4,4- trimethylcyclohex-2-en-1-yl)butan-1- one | 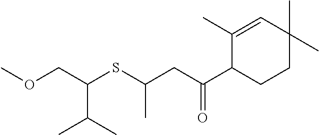 |
| 3-[(8-Ethoxy-4- tricyclo[5.2.1.02,6]decanyl)sulfanyl]-1- (2,4,4-trimethylcyclohex-2-en-1- yl)butan-1-one | 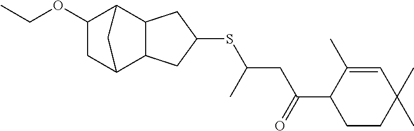 [SMILES: CCOC1CC2CC1C1CC(SC(C)CC(=O)C3CCC(C)(C)C=C3C)CC21] |
| 3-(2-Methoxy-1-methyl-ethyl)sulfanyl- 1-(2,4,4-trimethylcyclohex-2-en-1- yl)butan-1-one | 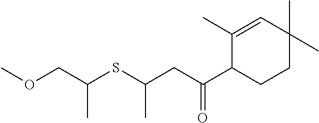 |
| 1-[2-[(8-Ethoxy-4- tricyclo[5.2.1.02,6]decanyl)sulfanyl]- 3,3-dimethyl-cyclohexyl]pent-4-en-1- one | 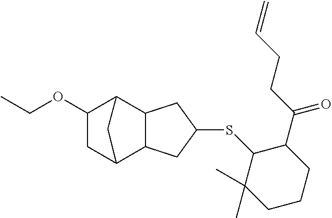 [SMILES: C=CCCC(=O)C1CCCC(C)(C)C1SC1CC2C3CC(OCC)C(C3)C2C1] |
| 1-[2-(1-Isopropyl-6-methoxy-4-methyl- hexyl)sulfanyl-3,3-dimethyl- cyclohexyl]pent-4-en-1-one | 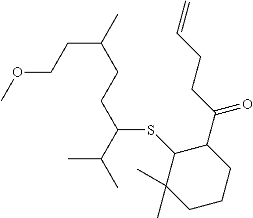 [SMILES: C=CCCC(=O)C1CCCC(C)(C)C1SC(CCC(C)CCOC)C(C)C] |
| 1-[2-(2-Methoxy-1-methyl- ethyl)sulfanyl-3,3-dimethyl- cyclohexyl]pent-4-en-1-one | 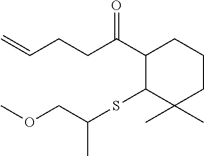 [SMILES: C=CCCC(=O)C1CCCC(C)(C)C1SC(C)COC] |
| 1-[2-(3-Methoxyoctylsulfanyl)-3,3- dimethyl-cyclohexyl]pent-4-en-1-one | 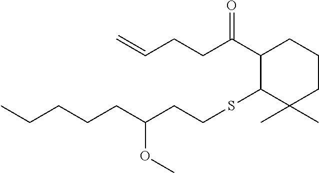 [SMILES: C=CCCC(=O)C1CCCC(C)(C)C1SCCC(CCCCC)OC] |
| 1-[2-(3-Methoxy-1,1-dimethyl- propyl)sulfanyl-5,5-dimethyl- cyclohexyl]pentan-1-one | 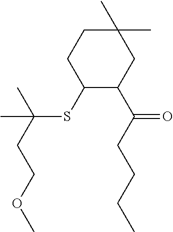 [SMILES: CCCCC(=O)C1CC(C)(C)CCC1SC(C)(C)CCOC] |
| 1-[2-[1-(2-Methoxyethyl)butylsulfanyl]- 5,5-dimethyl-cyclohexyl]pentan-1-one | 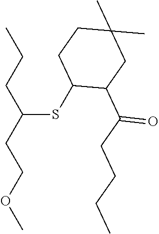 [SMILES: CCCCC(=O)C1CC(C)(C)CCC1SC(CCC)CCOC] |
| 1-[2-[1-(Hydroxymethyl)-1-methyl- butyl]sulfanyl-5,5-dimethyl- cyclohexyl]pentan-1-one | 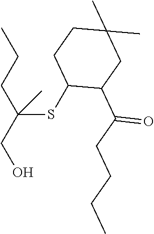 [SMILES: CCCCC(=O)C1CC(C)(C)CCC1SC(C)(CO)CCC] |
| 1-[2-(1-Isopropyl-4-methoxy-4-methyl- pentyl)sulfanyl-5,5-dimethyl- cyclohexyl]pentan-1-one | 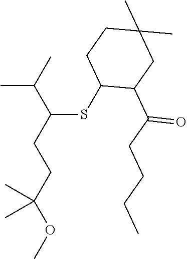 [SMILES: CCCCC(=O)C1CC(C)(C)CCC1SC(CCC(C)(C)OC)C(C)C] |
| 1-[3-(2-Methoxy-1-methyl- ethyl)sulfanyl-2,3-dihydrobenzofuran- 2-yl]ethanone | 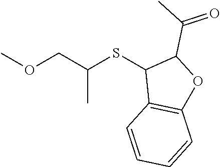 [SMILES: COCC(C)SC1c2ccccc2OC1C(C)=O] |
| 1-[3-[(8-Ethoxy-4- tricyclo[5.2.1.02,6]decanyl)sulfanyl]- 2,3-dihydrobenzofuran-2-yl]ethanone | 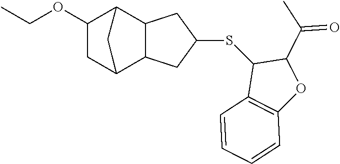 [SMILES: CCOC1CC2CC1C1CC(SC3c4ccccc4OC3C(C)=O)CC21] |
| 1-[3-[1-(Methoxymethyl)-2-methyl- propyl]sulfanyl-2,3- dihydrobenzofuran-2-yl]ethanone | 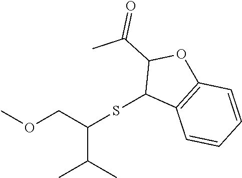 [SMILES: COCC(SC1c2ccccc2OC1C(C)=O)C(C)C] |
| 1-[3-[1-(Hydroxymethyl)-1-methyl- butyl]sulfanyl-2,3-dihydrobenzofuran- 2-yl]ethanone | 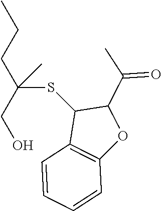 [SMILES: CCCC(C)(CO)SC1c2ccccc2OC1C(C)=O] |
| 1-[3-(3-Methoxy-1,1-dimethyl- propyl)sulfanyl-2,3- dihydrobenzofuran-2-yl]ethanone | 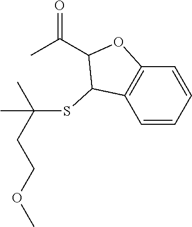 [SMILES: COCCC(C)(C)SC1c2ccccc2OC1C(C)=O] |
| 3-[1-(2- Methoxyethyl)butylsulfanyl]hexanal | 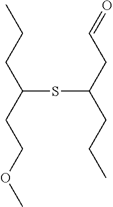 [SMILES: CCCC(CC=O)SC(CCC)CCOC] |
| 3-(1-Isopropyl-6-methoxy-4-methyl- hexyl)sulfanylhexanal | 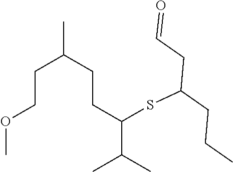 [SMILES: CCCC(CC=O)SC(CCC(C)CCOC)C(C)C] |
| 3-(3-Methoxyoctylsulfanyl)hexanal | 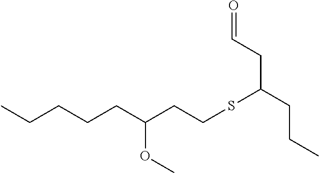 [SMILES: CCCCCC(CCSC(CC=O)CCC)OC] |
| 3-(2-Methoxy-1-methyl- ethyl)sulfanylhexanal | 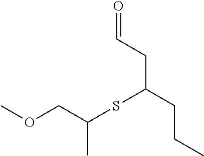 |
| 3-(1-Isopropyl-4-methoxy-4-methyl- pentyl)sulfanylundecanal | 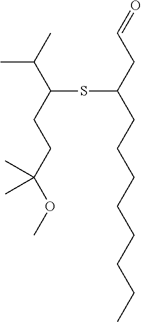 [SMILES: CCCCCCCCC(CC=O)SC(CCC(C)(C)OC)C(C)C] |
| 3-[(8-Ethoxy-4- tricyclo[5.2.1.02,6]decanyl)sulfanyl] undecanal | 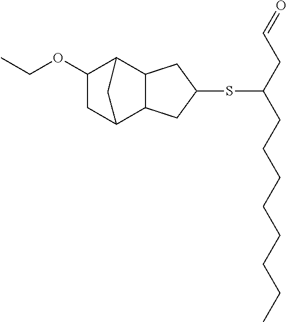 [SMILES: CCCCCCCCC(CC=O)SC1CC2C3CC(OCC)C(C3)C2C1] |
| 3-(3-Methoxyoctylsulfanyl)undecanal | 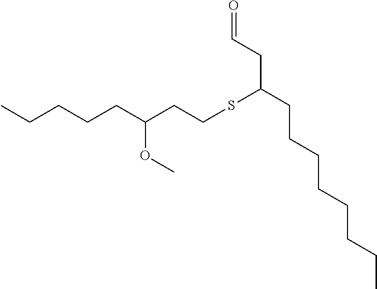 [SMILES: CCCCCCCCC(CC=O)SCCC(CCCCC)OC] |
| 3-(1-Isopropyl-6-methoxy-4-methyl- hexyl)sulfanylundecanal | 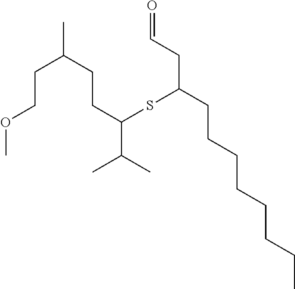 [SMILES: CCCCCCCCC(CC=O)SC(CCC(C)CCOC)C(C)C] |
| 3-(3-Methoxy-1,1-dimethyl- propyl)sulfanylundecanal | 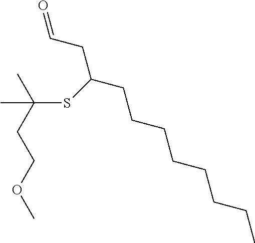 [SMILES: CCCCCCCCC(CC=O)SC(C)(C)CCOC] |
| 3-[1-(Hydroxymethyl)-1-methyl- butyl]sulfanyltridecanal | 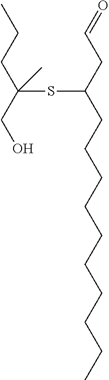 [SMILES: CCCCCCCCCCC(CC=O)SC(C)(CO)CCC] |
| 3-(3-Methoxyoctylsulfanyl)tridecanal | 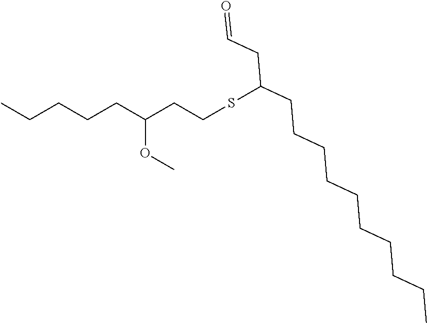 [SMILES: CCCCCCCCCCC(CC=O)SCCC(CCCCC)OC] |
| 3-(3-Methoxyoctylsulfanyl)-3,7- dimethyl-oct-6-enal | 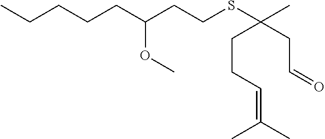 [SMILES: CCCCCC(CCSC(C)(CC=O)CCC=C(C)C)OC] |
| 3-[1-(Methoxymethyl)-2-methyl- propyl]sulfanyl-3,7-dimethyl-oct-6-enal | 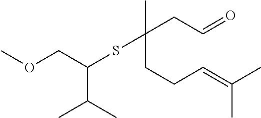 [SMILES: COCC(SC(C)(CC=O)CCC=C(C)C)C(C)C] |
| 3-[(8-Ethoxy-4- tricyclo[5.2.1.02,6]decanyl)sulfanyl]- 3,7-dimethyl-oct-6-enal | 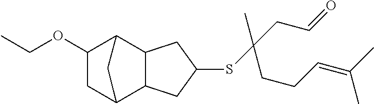 [SMILES: CCOC1CC2CC1C1CC(SC(C)(CC=O)CCC=C(C)C)CC21] |
| 3-[1-(2-Methoxyethyl)butylsulfanyl]- 3,7-dimethyl-oct-6-enal | 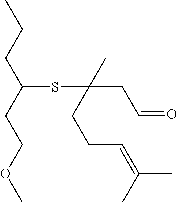 [SMILES: CCCC(CCOC)SC(C)(CC=O)CCC=C(C)C] |
| 3-((2-Methyl-1-(4-methyltetrahydro- 2H-pyran-2-yl)propyl)thio)-2- pentylcyclopentan-1-one | 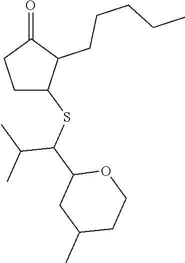 [SMILES: CCCCCC1C(=O)CCC1SC(C(C)C)C1CC(C)CCO1] |
| 5-Methyl-2-(2-((3-oxo-2- pentylcyclopentyl)thio)propan-2- yl)cyclohexan-1-one | 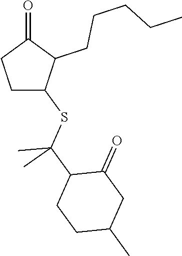 [SMILES: CCCCCC1C(=O)CCC1SC(C)(C)C1CCC(C)CC1=O] |
| 5-Methyl-2-[1-methyl-1-[1-methyl-3- oxo-3-(2,6,6-trimethylcyclohex-3-en-1- yl)propyl]sulfanyl-ethyl]cyclohexanone | 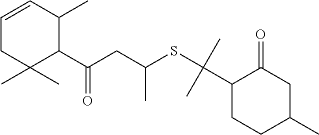 [SMILES: CC1CCC(C(C)(C)SC(C)CC(=O)C2C(C)C=CCC2(C)C)C(=O)C1] |

According to a particularly preferred embodiment, the compound of formula (I) according to the invention is not

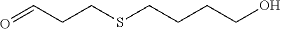

Another aspect of the present invention relates to a fragrance substance mixture, preferably perfume oil, comprising or consisting of the following components:
(i) one or more compound(s) of formula (I) as defined herein,
and
(ii) one or more further fragrance substances.

The one or more further fragrance substances are different from the compounds of formula (I) according to the invention.

Examples of further fragrance substances that can be advantageously combined with the compounds of formula (I) as defined herein within the scope of the present invention are known to the skilled person and can be found, for example, in S. Arctander, Perfume and Flavor Materials, Vol. I and II, Montclair, N. J. 1969, Eigenverlag, or K. Bauer et al., Common Fragrance and Flavor Materials, 4th Edition, Wiley-VCH, Weinheim 2001.

According to a preferred embodiment of the fragrance substance mixture according to the invention, preferably perfume oil, the proportion of the total amount of compound(s) of formula (I) as defined herein in the fragrance substance mixture is 0.1 to 3% by weight, preferably 0.5 to 2.5% by weight, particularly preferably 1 to 2% by weight, based on the total mass of the fragrance substance mixture.

Another aspect of the present invention relates to a method for producing a fragrance substance mixture, preferably a perfume oil, as defined above, comprising or consisting of the following step:

Mixing of one or more compound(s) of formula (I) as defined herein with one or more further fragrance substances.

Furthermore, fragrance substance mixtures, preferably perfume oils, according to the invention may be adsorbed on a carrier which provides both a fine distribution of the fragrance substance mixture, preferably perfume oil, in the perfumed product and a controlled release during application. Such carriers may be porous inorganic materials such as light sulphate, silica gels, zeolites, gypsums, clays, clay granules, gas concrete, etc. or organic materials such as wood, cellulose-based materials, sugar, dextrins (e.g. maltodextrin) or plastics such as PVC, polyvinyl acetates or polyurethanes. The combination of a fragrance substance mixture, preferably perfume oil, according to the invention and a carrier is also to be understood as a fragrance substance mixture, preferably perfume oil, of the invention or may be presented as a perfumed product according to the invention (as described below).

Fragrance substance mixtures, preferably perfume oils, or perfumed products of the invention (as described below) may also be microencapsulated, spray-dried, or may be provided as inclusion complexes or as extrusion products and, in the case of a fragrance substance mixture, preferably perfume oil, may be added in this form e.g. to a perfumed product to be perfumed (as described below).

If necessary, the properties of such modified mixtures or products can be further optimized by so-called “coating” with suitable materials with a view to a more targeted release of fragrance, preferably using wax-like plastics such as polyvinyl alcohol. The resulting products in turn represent products of the invention.

Microencapsulation can, for example, be achieved by the so-called coacervation method using capsule materials such as polyurethane-like substances or soft gelatin.

Spray-dried products are preferably produced by spray-drying an emulsion or dispersion containing the fragrance substance mixtures, preferably perfume oils, according to the invention, whereby modified starches, proteins, dextrin and vegetable gums can be used as carriers.

Inclusion complexes can be produced e.g. by incorporating dispersions of the fragrance substance mixtures, preferably perfume oils, according to the invention and cyclodextrins or urea derivatives into a suitable solvent, e.g. water.

Extrusion products can be obtained, e.g., by using the fragrance substance mixtures, preferably perfume oils, according to the invention with a suitable waxy substance and by extrusion followed by solidification, if necessary, in a suitable solvent, e.g. isopropanol.

Another aspect of the present invention relates to a perfumed product comprising one or more compound(s) of formula (I) as defined herein or a fragrance substance mixture, preferably a perfume oil, as defined herein, preferably in a sensorially effective amount.

Advantageously, the one or more compound(s) of formula (I) according to the invention positively impart or modify the odour of a perfumed product to which said compound(s) of formula (I) are added.

According to a preferred embodiment, the perfumed product is selected from the group consisting of perfume extracts, eau de parfums, eau de toilettes, aftershaves, eau de colognes, pre-shave products, splash colognes, perfumed refreshing cloths, acidic, alkaline and neutral detergents, textile fresheners, ironing aids, liquid detergents, powdered detergents, laundry pre-treatment agents, fabric softeners, washing soaps, washing tablets, disinfectants, surface disinfectants, air fresheners, aerosol sprays, waxes and polishes, personal care products, hand creams and lotions, foot creams and lotions, depilatory creams and lotions, aftershave creams and lotions, tanning creams and lotions, hair care products, deodorants and antiperspirants, decorative cosmetic products, candles, lamp oils, incense sticks, insecticides, repellents, and fuels, most preferably is a fabric softener.

According to a preferred embodiment of the perfumed product according to the invention, the proportion of the total amount of fragrance substance mixture as defined herein, preferably perfume oil, in the perfumed product is 0.1 to 3% by weight, preferably 0.5 to 2.5% by weight, particularly preferably 1 to 2% by weight, based on the total mass of the perfumed product.

Another aspect of the present invention relates to a method for producing a perfumed product, preferably a perfumed product as defined herein, comprising or consisting of the following steps:
(i) Providing one or more compound(s) of formula (I) as defined herein or a fragrance substance mixture, preferably a perfume oil, as defined herein,
(ii) providing one or more further components of the perfumed product to be produced,
   and
(iii) contacting or mixing the further components provided in step (ii) with a sensorially effective amount of the components provided in step (i).

Another aspect of the present invention relates to the use of a compound of formula (I) as defined herein as a precursor for the (controlled) release of one, two, three or more different pleasantly smelling fragrance substances, preferably wherein the two or more different fragrance substances are released and/or sensorially perceived at the same or different, preferably different, point(s) in time (if two or more different fragrance substances are released).

Another aspect of the present invention relates to the use of a compound of formula (I) as defined herein as a precursor for the (controlled) release of a pleasantly smelling thiol and of a pleasantly smelling α,β-unsaturated aldehyde or ketone, preferably wherein the thiol and the α,β-unsaturated aldehyde or ketone are released and/or sensorially perceived at the same or different, preferably different, point(s) in time.

Another aspect of the present invention relates to the use of a compound of formula (I) as defined herein as a precursor for the (controlled) release of a (pleasantly smelling) thiol selected from the group consisting of

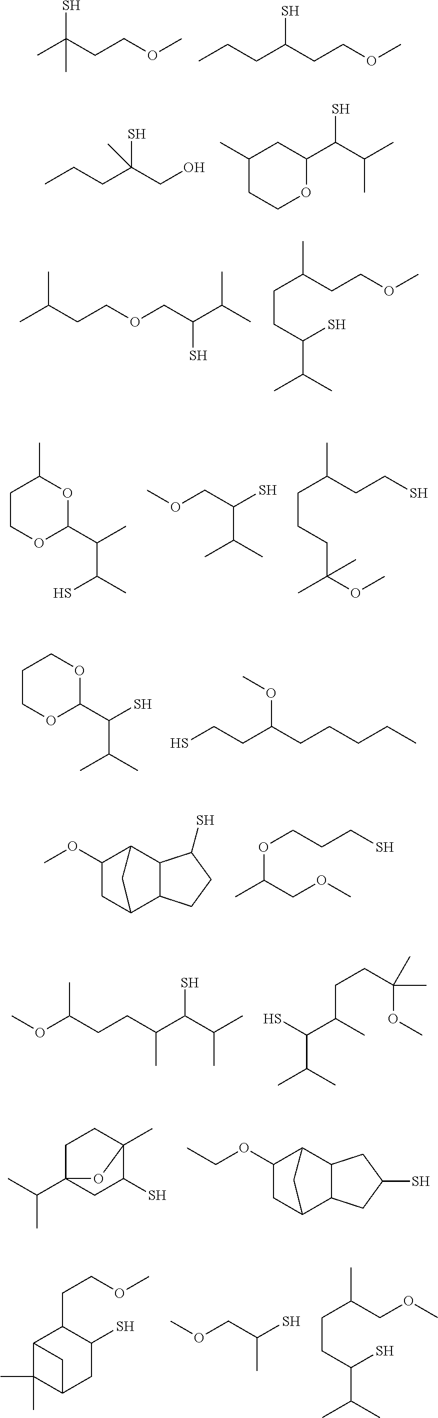

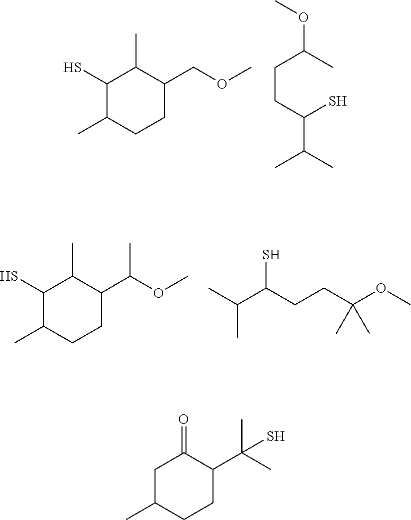

and/or, preferably and, of a (pleasantly smelling) α,β-unsaturated aldehyde or ketone selected from the group consisting of

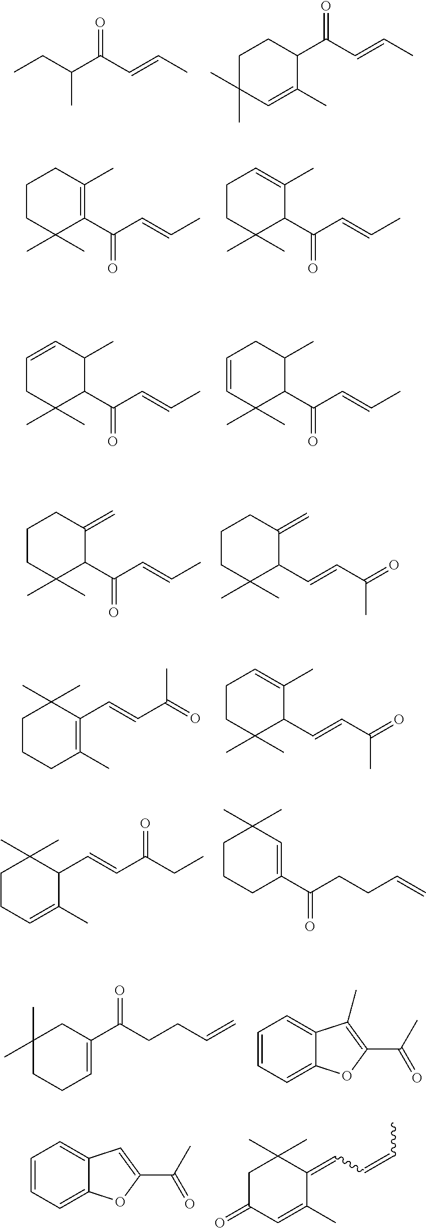

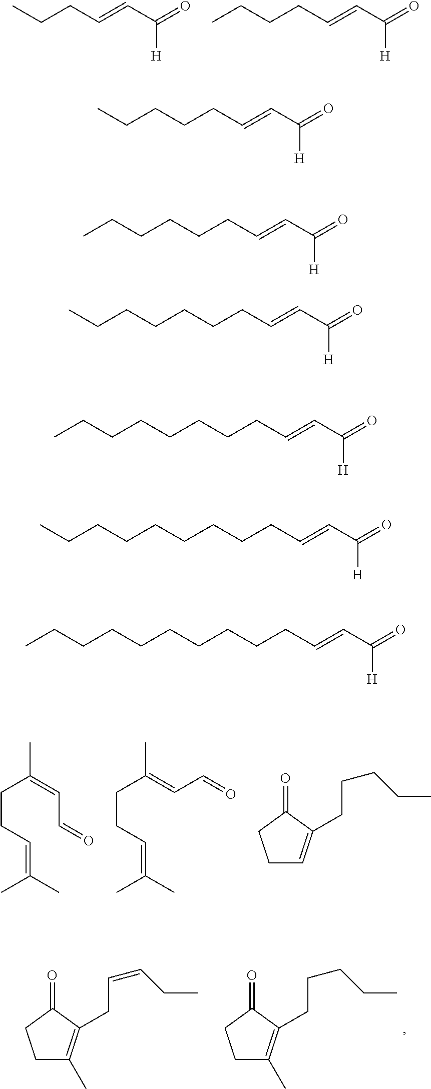

preferably wherein the thiol and the α,β-unsaturated aldehyde or ketone are released and/or sensorially perceived at the same or different, preferably different, point(s) in time.

Another aspect of the present invention relates to the use of a compound of formula (I), wherein residue

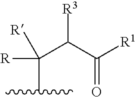

of the compound of formula (I) is selected from the group consisting of

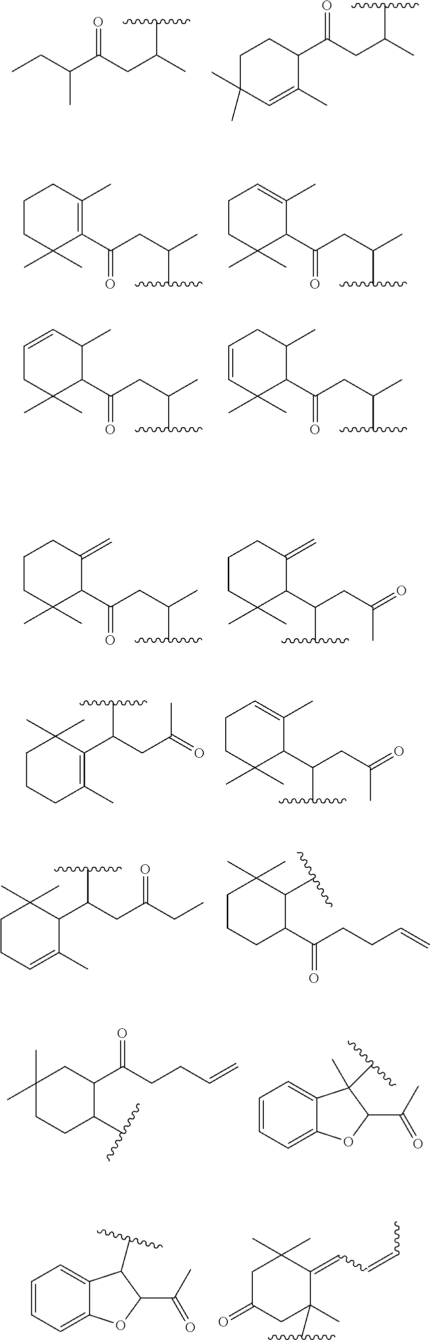

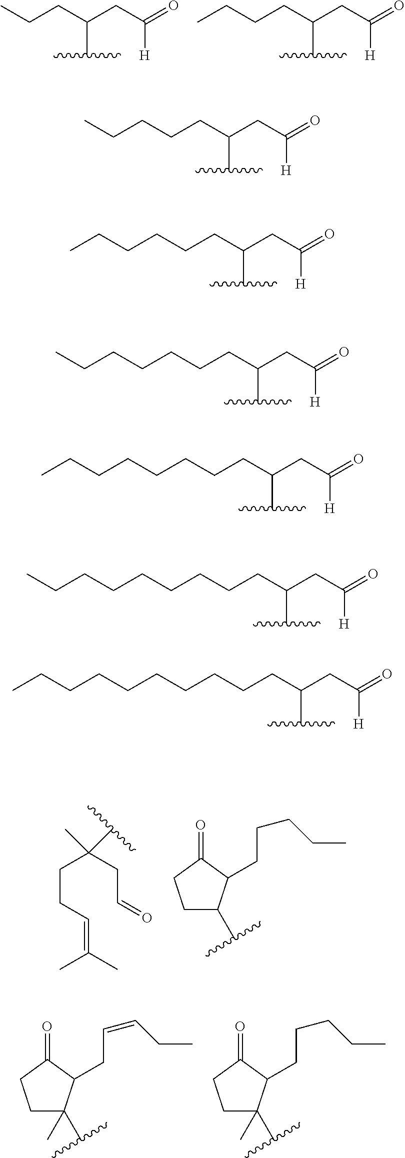

and/or, preferably and, wherein residue R² of the compound of formula (I) is selected from the group consisting of

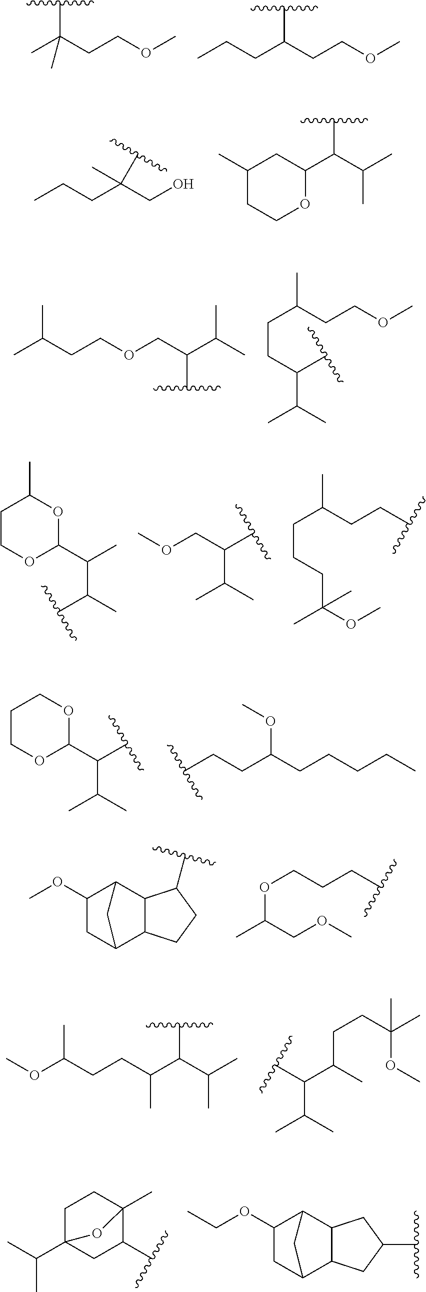

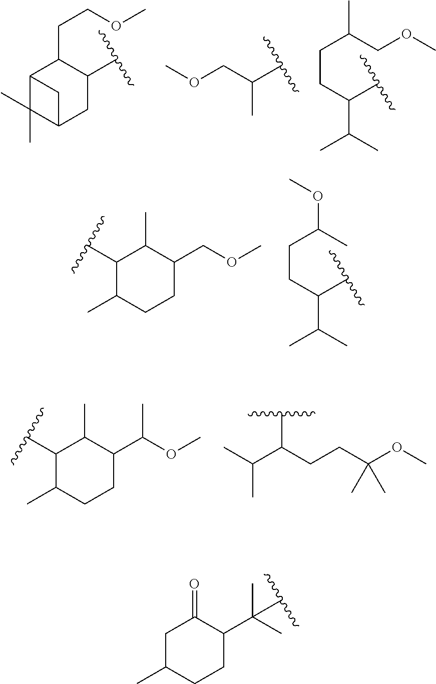

as a precursor for the (controlled) release of a (pleasantly smelling) thiol selected from the group consisting of

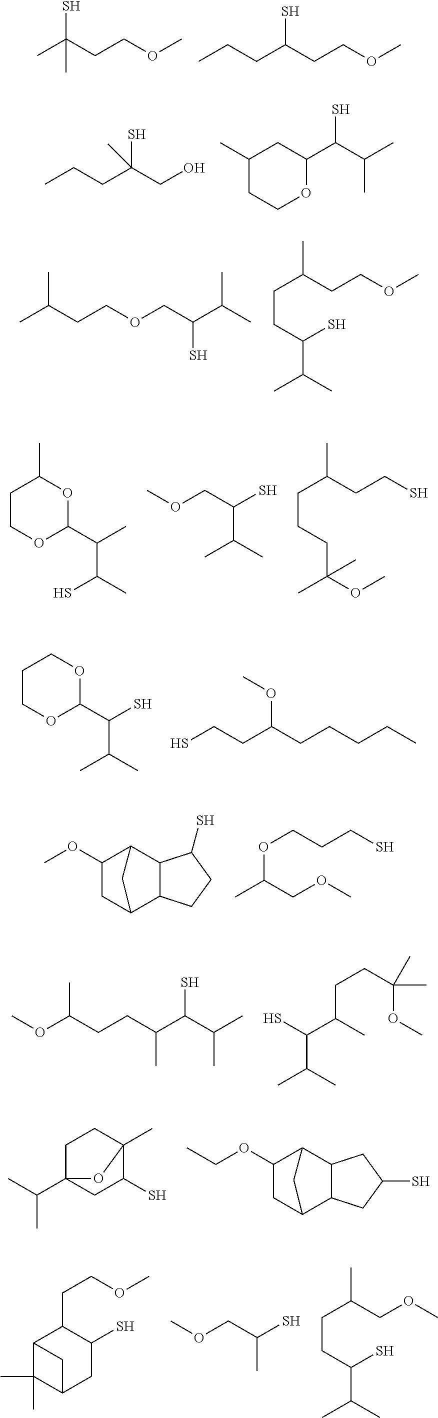

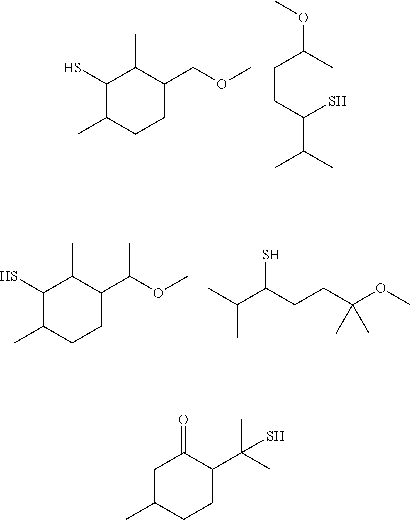

and/or, preferably and, of a (pleasantly smelling) α,β-unsaturated aldehyde or ketone selected from the group consisting of

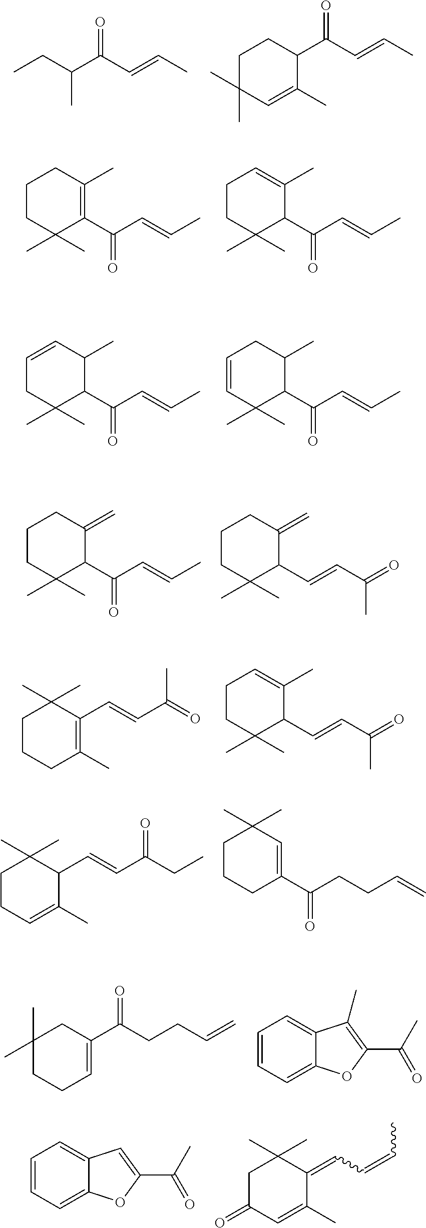

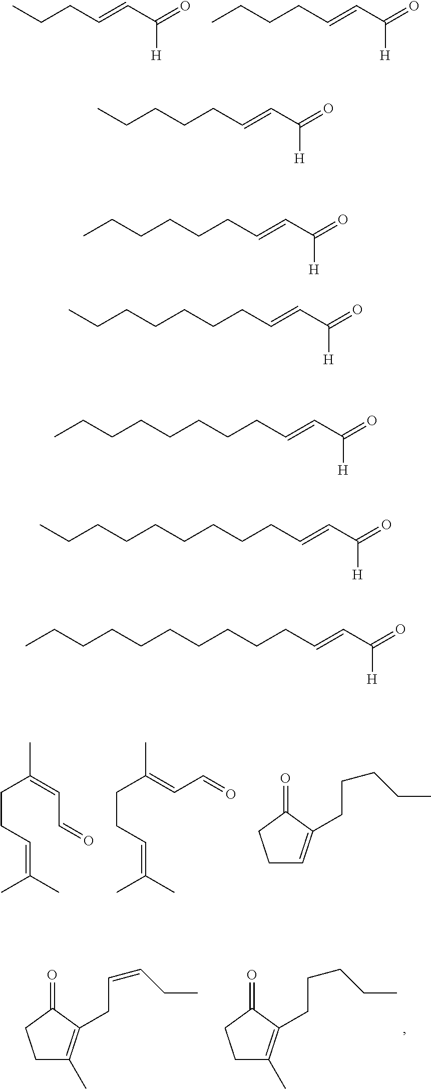

preferably wherein the thiol and the α,β-unsaturated aldehyde or ketone are released and/or sensorially perceived at the same or different, preferably different, point(s) in time.

The thiols and α,β-unsaturated aldehydes or ketones released by the compounds of formula (I) according to the invention and/or used for the formation of a compound of formula (I) according to the invention may be in the form of a pure diastereomer or stereoisomer or in the form of any mixture of said isomers.

According to a preferred embodiment of the use according to the invention, the thiol that is released from the compound of formula (I) as defined herein is released and/or sensorially perceived before the α,β-unsaturated aldehyde or ketone that is released from the (same) compound of formula (I) as defined herein.

According to an alternative embodiment of the use according to the invention, the α,β-unsaturated aldehyde or ketone that is released from the compound of formula (I) as defined herein is released and/or sensorially perceived before the thiol that is released from the (same) compound of formula (I) as defined herein.

According to a further alternative embodiment of the use according to the invention, the thiol that is released from the compound of formula (I) as defined herein and the α,β-unsaturated aldehyde or ketone that is released from the (same) compound of formula (I) as defined herein are released and/or sensorially perceived at the same time.

Preferably, a pleasantly smelling thiol has one or more odour notes selected from the group consisting of grapefruit, cassis, onion, fruity, exotic fruit, guava, breath of smoker, sulphur, (burnt) plastic, (minty) green, rose oxide, green pepper, liver sausage, buchu (Asian chives), gasoline, juicy, natural, cat grass, meat broth (“Fleischbrühe”), cheesy, camembert, cheese in the fridge, fresh, hot rubber, petrol, violet, blackcurrant, catnip (grass), zestral, anisic, mushroom, soup, leek, agrumen, peach, meat, pine, mango, durian, jackfruit, artichoke, and apricot,

more preferably a pleasantly smelling thiol has one or more odour notes selected from the group consisting of grapefruit, cassis, onion, fruity, exotic fruit, guava, sulphur, (minty) green, rose oxide, green pepper, liver sausage, buchu (Asian chives), juicy, natural, cat grass, meat broth (“Fleischbrühe”), cheesy, camembert, fresh, violet, blackcurrant, catnip (grass), zestral, anisic, mushroom, soup, leek, agrumen, peach, meat, pine, mango, durian, jackfruit, artichoke, and apricot.

Another aspect of the present invention relates to a method for the (controlled) release of one, two, three or more different pleasantly smelling fragrance substances comprising or consisting of the following steps:
(a) Providing a compound of formula (I) as defined herein or a fragrance substance mixture as defined herein (comprising one or more compound(s) of formula (I) as defined herein) or a perfumed product as defined herein (comprising one or more compound(s) of formula (I) as defined herein),
(b) optionally, applying or introducing the compound of formula (I) or the fragrance substance mixture or the perfumed product of step (a) to hair or skin or textile fibres or to a surface to be perfumed, or into ambient air to be perfumed, preferably in a sensorially effective amount, and
(c) releasing one, two, three or more different pleasantly smelling fragrance substances from the compound of formula (I), preferably wherein the two or more different pleasantly smelling fragrance substances are released and/or sensorially perceived at the same or different, preferably different, point(s) in time (if two or more different fragrance substances are released).

Another aspect of the present invention relates to a method for the (controlled) release of a pleasantly smelling thiol and of a pleasantly smelling α,β-unsaturated aldehyde or ketone comprising or consisting of the following steps:
(a) Providing a compound of formula (I) as defined herein or a fragrance substance mixture as defined herein (comprising one or more compound(s) of formula (I) as defined herein) or a perfumed product as defined herein (comprising one or more compound(s) of formula (I) as defined herein),
(b) optionally, applying or introducing the compound of formula (I) or the fragrance substance mixture or the perfumed product of step (a) to hair or skin or textile fibres or to a surface to be perfumed, or into ambient air to be perfumed, preferably in a sensorially effective amount,
(c) releasing the pleasantly smelling thiol and the pleasantly smelling α,β-unsaturated aldehyde or ketone from the compound of formula (I), preferably wherein the pleasantly smelling thiol and the pleasantly smelling α,β-unsaturated aldehyde or ketone are released and/or sensorially perceived at the same or different, preferably different, point(s) in time.

According to a preferred embodiment of the method according to the invention, the thiol that is released from the compound of formula (I) as defined herein in step (c) is released and/or sensorially perceived before the α,β-unsaturated aldehyde or ketone that is released from the (same) compound of formula (I) as defined herein in step (c).

According to an alternative embodiment of the method according to the invention, the α,β-unsaturated aldehyde or ketone that is released from the compound of formula (I) as defined herein in step (c) is released and/or sensorially perceived before the thiol that is released from the (same) compound of formula (I) as defined herein in step (c).

According to a further alternative embodiment of the method according to the invention, the thiol that is released from the compound of formula (I) as defined herein in step (c) and the α,β-unsaturated aldehyde or ketone that is released from the (same) compound of formula (I) as defined herein in step (c) are released and/or sensorially perceived at the same time.

Another aspect of the present invention relates to a method for the (controlled) release of a (pleasantly smelling) thiol selected from the group consisting of

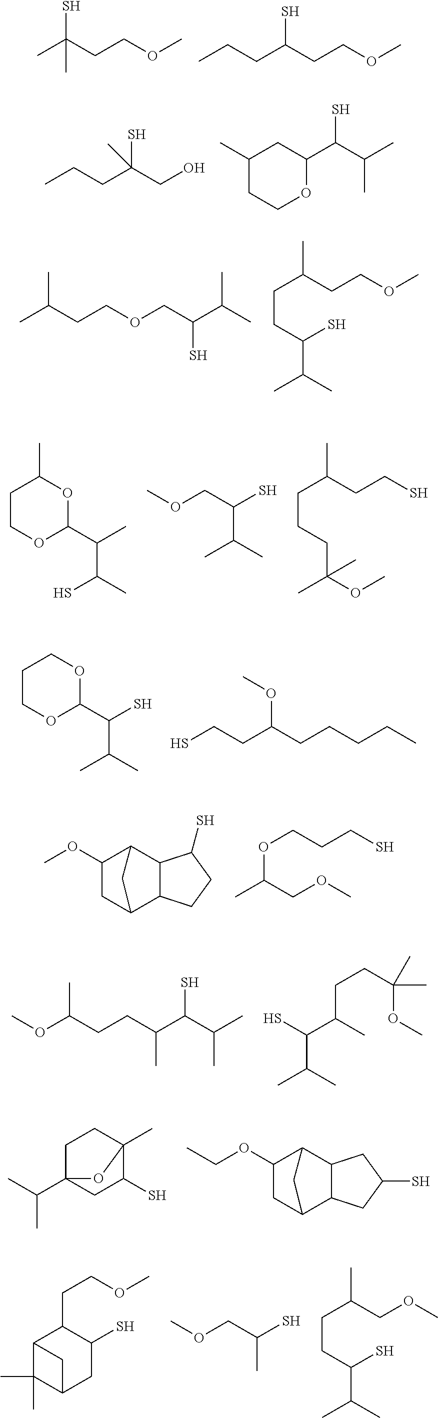

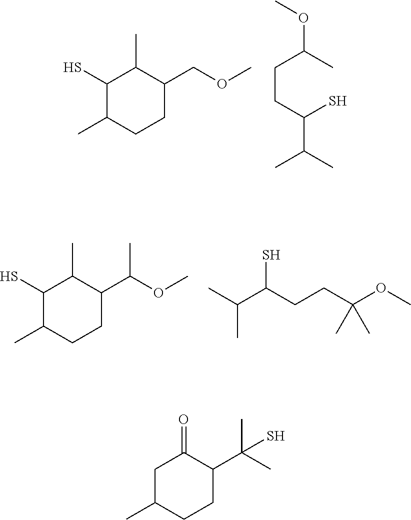

and/or, preferably and, of a (pleasantly smelling) α,β-unsaturated aldehyde or ketone selected from the group consisting of

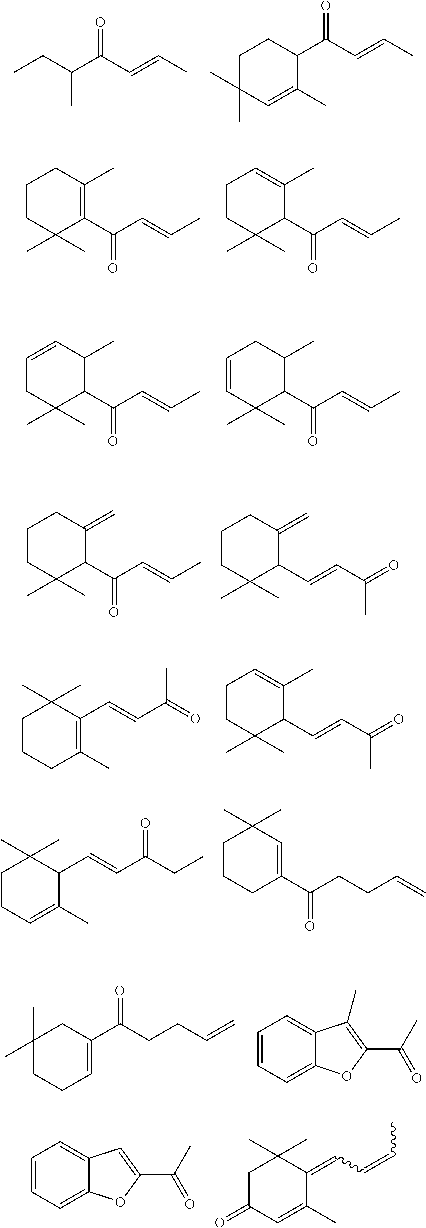

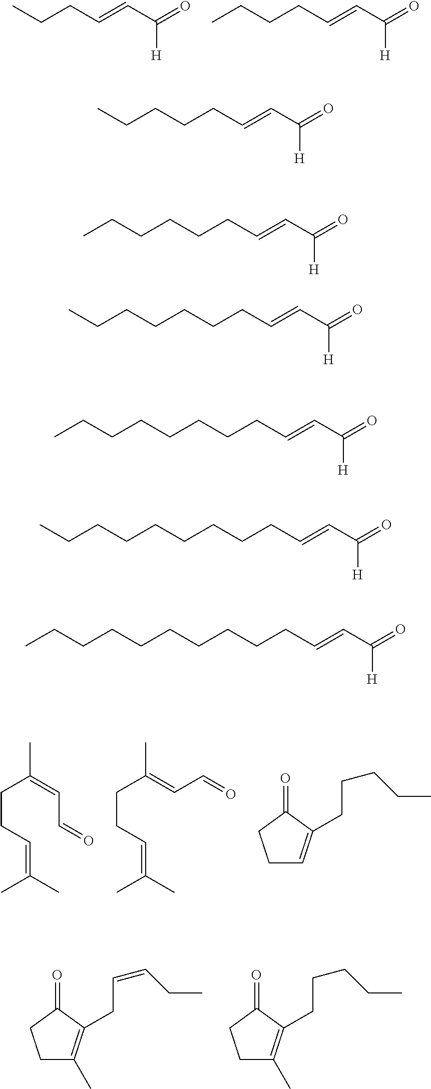

comprising or consisting of the following steps:
(a) Providing a compound of formula (I) (or a fragrance substance mixture or a perfumed product comprising a compound of formula (I) as defined above), wherein residue

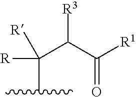

of the compound of formula (I) is selected from the group consisting of

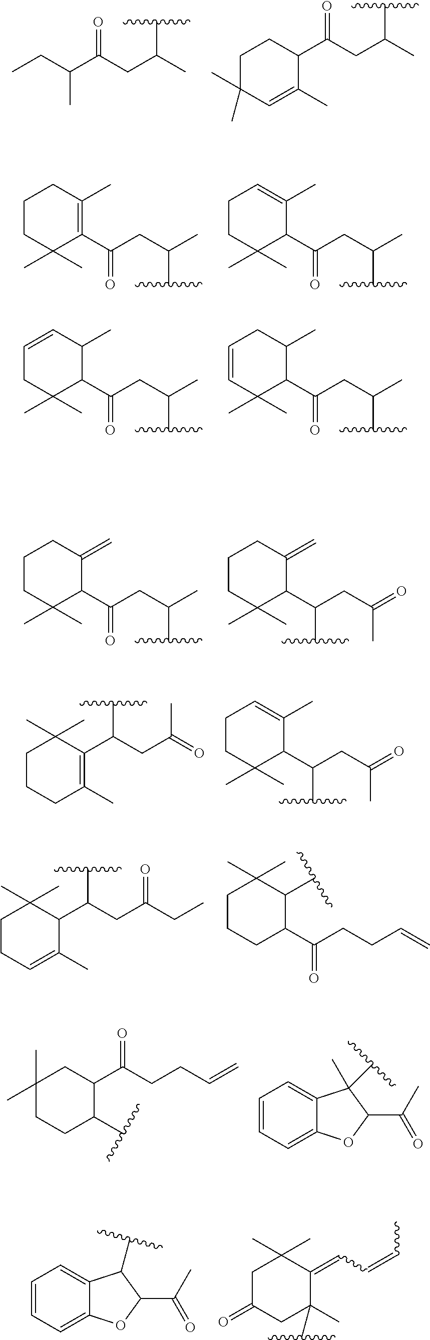

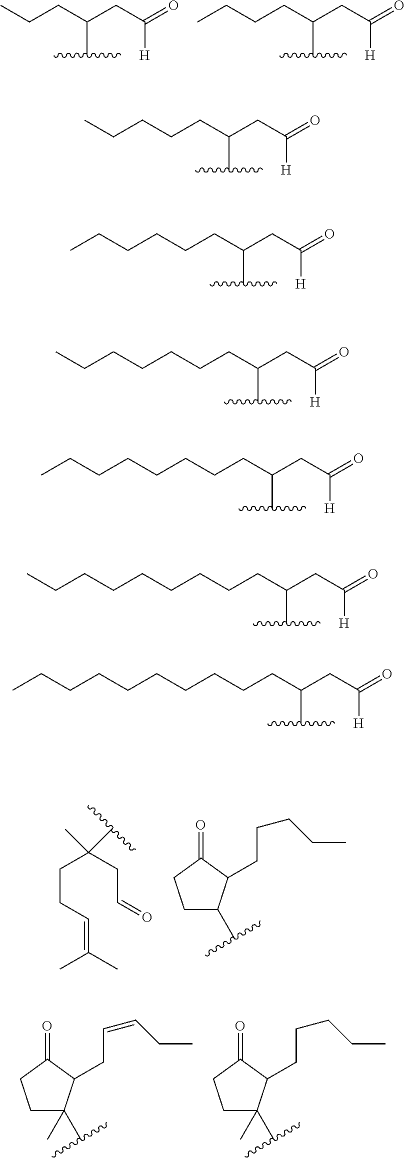

and/or, preferably and, wherein residue R² of the compound of formula (I) is selected from the group consisting of

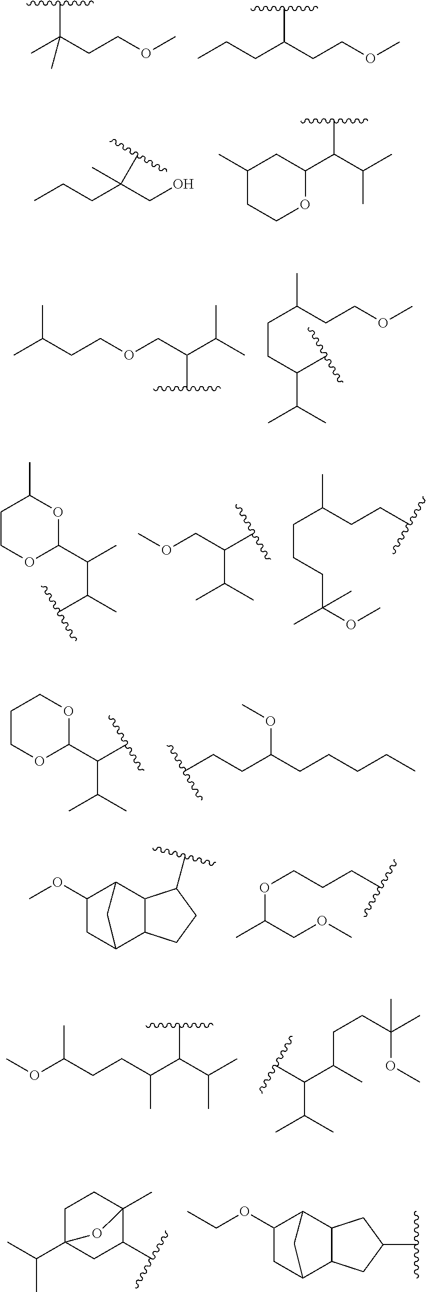

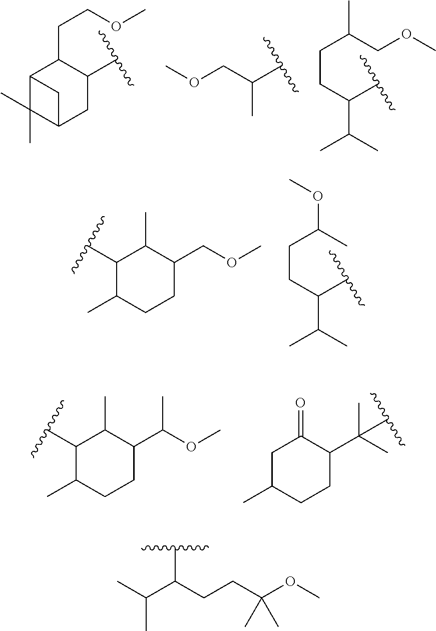

(b) optionally, applying or introducing the compound of formula (I) of step (a) (or the fragrance substance mixture or the perfumed product comprising a compound of formula (I) of step (a)) to hair or skin or textile fibres or to a surface to be perfumed, or into ambient air to be perfumed, preferably in a sensorially effective amount,
(c) releasing the (pleasantly smelling) thiol and/or the (pleasantly smelling) α,β-unsaturated aldehyde or ketone from the compound of formula (I), preferably wherein the (pleasantly smelling) thiol and the (pleasantly smelling) α,β-unsaturated aldehyde or ketone are released and/or sensorially perceived at the same or different, preferably different, point(s) in time.

Another aspect of the present invention relates to a thiol selected from the group consisting of

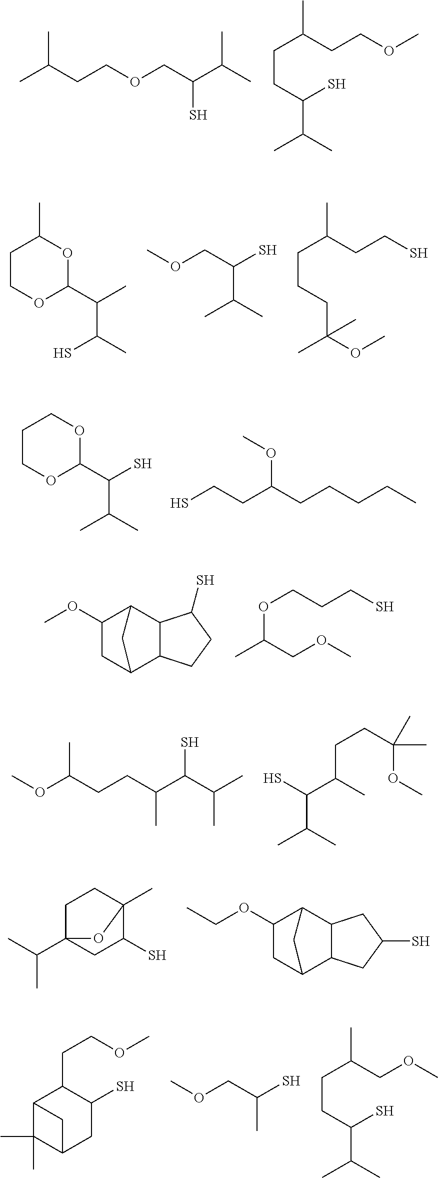

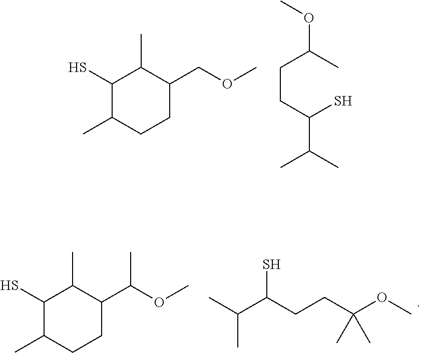

During the studies underlying the present invention, it was surprisingly found that the thiols as defined above can serve as pleasantly smelling, high impact fragrances, preferably after release from a compound of formula (I) according to the invention.

The thiols according to the invention can be obtained by applying the procedures disclosed in WO 2019/037862 A1.

An odour description of the thiols according to the invention can be found in Table 2 above.

Another aspect of the present invention thus relates to the use of one or more thiol(s) according to the invention as a fragrance substance.

Another aspect of the present invention relates to a method for perfuming hair, skin, textile fibres, surfaces and/or ambient air comprising or consisting of the following steps:
(a) Providing
   (i) one or more thiol(s) as defined herein and preferably also a surfactant or a surfactant mixture, or
   (ii) one or more compound(s) of formula (I) as defined herein and preferably also a surfactant or a surfactant mixture, or
   (iii) a fragrance substance mixture as defined herein, preferably containing a surfactant or a surfactant mixture, or
   (iv) a perfumed product as defined herein, preferably containing a surfactant or a surfactant mixture,
   and
(b) applying or introducing the (i) thiol(s) and preferably also a surfactant or a surfactant mixture or (ii) compound(s) and preferably also a surfactant or a surfactant mixture or the (ii) fragrance substance mixture or the (iii) perfumed product of step (a) to the hair or skin or textile fibres or surface to be perfumed, or into the ambient air to be perfumed, in a sensorially effective amount.

Another aspect of the present invention relates to the use of a compound of formula (I) as defined herein or of a fragrance substance mixture, preferably perfume oil, as defined herein as a perfuming ingredient.

Another aspect of the present invention relates to the use a compound of formula (I) as defined herein or of a fragrance substance mixture, preferably perfume oil, as defined herein for perfuming hair, skin, textile fibres, surfaces and/or ambient air.

Another aspect of the present invention relates to a fragrance substance mixture, preferably perfume oil, as defined herein, wherein the amount of compound(s) of formula (I) is sufficient
(a) to mask or to reduce the or one or several unpleasant odour impressions of another fragrance substance in the fragrance substance mixture,
and/or
(b) to enhance the or one or several pleasant odour impressions of another fragrance substance in the fragrance substance mixture.

Another aspect of the present invention relates to the use of one or more compound(s) of formula (I) as defined herein or of a fragrance substance mixture, preferably perfume oil, as defined herein
(a) for masking or reducing the or one or several unpleasant odour impressions of one or several unpleasantly smelling substances,
and/or
(b) for enhancing the or one or several pleasant odour impressions of one or several pleasantly smelling substances.

Another aspect of the present invention relates to a method
(a) for masking or reducing the or one or several unpleasant odour impressions of one or several unpleasantly smelling substances,
and/or
(b) for enhancing the or one or several pleasant odour impressions of one or several pleasantly smelling substances, comprising the following step:
Mixing the unpleasantly and/or pleasantly smelling substances with one or more compound(s) of formula (I) as defined herein or with a fragrance substance mixture, preferably perfume oil, as defined herein, wherein the amount of the one or more compound(s) of formula (I) as defined herein or of the fragrance substance mixture, preferably perfume oil, as defined herein is sufficient to (a) reduce or to mask the unpleasant odour impression(s) of the unpleasantly smelling substance(s) and/or (b) enhance the pleasant odour impression(s) of the pleasantly smelling substance(s).

What has been stated herein in connection with the fragrance substance mixtures, perfumed products, methods, uses and thiols according to the invention applies accordingly to preferred embodiments of the compounds of formula (I) according to the invention, and vice versa.

In the following, the invention is explained in more detail using examples.

### EXAMPLES

### Synthesis Procedures of Precursors According to the Invention

### 1. Synthesis of Precursor 3-[2-methyl-1-(4-methyltetrahydropyran-2-yl)propyl]sulfanyl-1-(2,6,6-trimethylcyclohex-3-en-1-yl) butan-1-one (Compound of Formula (I) According to the Invention)

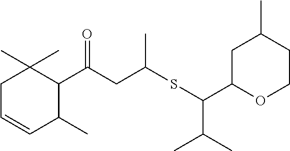 [SMILES: CC1CCOC(C(SC(C)CC(=O)C2C(C)C=CCC2(C)C)C(C)C)C1]

In a 500 ml stirring apparatus with magnetic stirrer, reflux condenser, thermometer, dropping funnel and ice bath, 5.8 g of 1,1,3,3-tetramethylguanidine, 9.4 g of 2-methyl-1-(4-methyltetrahydropyran-2-yl)propanethiol and 200 ml of tetrahydrofuran were added under a nitrogen atmosphere at 0° C. At 0-5° C., 8.1 g of delta-damascone were added dropwise within 1 hour. For post-reaction, stirring was continued for 6 hours at 0-5° C. The mixture was washed twice with saturated sodium bicarbonate solution at room temperature and then the solvents were evaporated on the rotary evaporator. 16.8 g crude yield of 3-[2-methyl-1-(4-methyltetrahydropyran-2-yl)propyl]sulfanyl-1-(2,6,6-trimethyl-cyclohex-3-en-1-yl) butan-1-one, with a GC content of 90%, were obtained. For further purification, the crude yield was chromatographed over a silica gel column with hexane:diethyl ether (10:1) as eluent. 4.0 g of 3-[2-methyl-1-(4-methyltetrahydropyran-2-yl)propyl]sulfanyl-1-(2,6,6-trimethylcyclohex-3-en-1-yl) butan-1-one, with a GC content of 94%, were obtained.

Mass spectrometric analysis of product:

m/z: 99 (100), 123 (35), 81 (35), 43 (33), 69 (27), 115 (24), 55 (18), 225 (17), 41 (17), 192 (16) 2. Synthesis of precursor 3-[2-methyl-1-(4-methyltetrahydropyran-2-yl) propyl]-sulfanyldecanal (compound of formula (I) according to the invention)

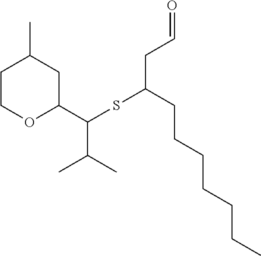 [SMILES: CCCCCCCC(CC=O)SC(C(C)C)C1CC(C)CCO1]

In a 500 ml stirring apparatus with magnetic stirrer, reflux condenser, thermometer, dropping funnel and ice bath, 5.8 g of 1,1,3,3-tetramethylguanidine, 9.4 g of 2-methyl-1-(4-methyltetrahydropyran-2-yl)propanethiol and 200 ml of tetrahydrofuran were placed under a nitrogen atmosphere at 0° C. At 0-5° C., 6.4 g of 2-(E)-decenal were added dropwise within 1 hour. For post-reaction, stirring was continued for 6 hours at 0-5° C. The mixture was washed twice with saturated sodium bicarbonate solution at room temperature and then the solvents were evaporated on the rotary evaporator. 14.6 g crude yield of 3-[2-methyl-1-(4-methyltetrahydropyran-2-yl)propyl]sulfanyldecanal, with a GC content of 76%, were obtained. For further purification, the crude yield was chromatographed over a silica gel column with hexane:diethyl ether (10:1) as eluent. 4.2 g of 3-[2-methyl-1-(4-methyltetrahydropyran-2-yl)propyl]sulfanyldecanal, with a GC content of 90%, were obtained.

Mass spectrometric analysis of product:

m/z: 99 (100), 43 (23), 55 (17), 81 (13), 41 (13), 69 (11), 57 (10), 98 (8), 29 (7), 100 (7)

### 3. Synthesis of Precursor 3-[2-methyl-1-(4-methyltetrahydropyran-2-yl)propyl]sulfanylhexanal (Compound of Formula (I) According to the Invention)

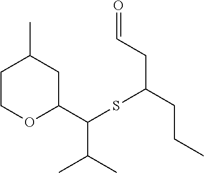 [SMILES: CCCC(CC=O)SC(C(C)C)C1CC(C)CCO1]

In a 500 mL stirring apparatus with magnetic stirrer, reflux condenser, thermometer, dropping funnel and ice bath, 12.4 g of 1,1,3,3-tetramethylguanidine, 20.4 g of 2-methyl-1-(4-methyltetrahydropyran-2-yl)propane-1-thiol and tetrahydrofuran (400 mL) were added under a nitrogen atmosphere at 0° C. At 0-5° C., 12.7 g of (E)-hex-2-enal were added dropwise within 1 hour. For post-reaction, stirring was continued for 6 hours at 0-5° C. The mixture was washed twice with saturated sodium bicarbonate solution at room temperature and then the solvents were evaporated on the rotary evaporator. 30.9 g crude of 3-[2-methyl-1-(4-methyltetrahydropyran-2-yl)propyl]sulfanyl-hexanal were obtained. For further purification, the crude yield was chromatographed over a silica gel column with hexane:diethyl ether (10:1) as eluent. 26.8 g of 3-[2-methyl-1-(4-methyltetrahydropyran-2-yl)propyl]sulfanylhexanal, with a GC content of 90%, were obtained.

Mass spectrometric analysis of product:

m/z: 99 (100), 43 (24), 55 (15), 41 (13), 81 (12), 69 (12), 100 (6), 57 (6), 170 (6), 29 (5), 98 (5).

### 4. Synthesis of Precursor 3-[2-methyl-1-(4-methyltetrahydropyran-2-yl)propyl]sulfanyl-1-(2,6,6-trimethyl-1-cyclohexen-1-yl)-3-butan-2-one (Compound of Formula (I) According to the Invention)

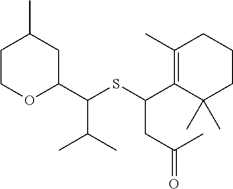 [SMILES: CC(=O)CC(SC(C(C)C)C1CC(C)CCO1)C1=C(C)CCCC1(C)C]

In a 50 mL stirring apparatus with magnetic stirrer, reflux condenser, thermometer, dropping funnel and ice bath, 0.02 g of 1,1,3,3-tetramethylguanidine, 14.1 g of 2-methyl-1-(4-methyltetrahydropyran-2-yl)propane-1-thiol and tetrahydrofuran (40 mL) were added under a nitrogen atmosphere at 0° C. At 0-5° C., 14.4 g of (E)-[4]-(2,6,6-trimethylcyclohex-1-en-1-yl) but-3-en-2-one were added dropwise within 1 hour. For post-reaction, stirring was continued for 6 hours at 0-5° C. The mixture was washed twice with saturated sodium bicarbonate solution at room temperature and then the solvents were evaporated on the rotary evaporator. 28.5 g crude of 3-[2-methyl-1-(4-methyltetrahydropyran-2-yl)propyl]sulfanyl-1-(2,6,6-trimethyl-1-cyclohexen-1-yl)-3-butan-2-one were obtained. For further purification, the crude yield was chromatographed over a silica gel column with hexane:diethyl ether (10:1) as eluent. 18.9 g of 3-[2-methyl-1-(4-methyltetrahydropyran-2-yl)propyl]sulfanyl-1-(2,6,6-trimethyl-1-cyclohexen-1-yl)-3-butan-2-one, with a GC content of 90%, were obtained.

Mass spectrometric analysis of product:

m/z: 43 (100), 99 (78), 177 (35), 175 (33), 133 (29), 41 (29), 135 (27), 55 (24), 81 (19), 69 (18), 193 (15), 149 (15), 192 (12), 93 (12), 29 (11), 91 (11), 79 (10).

### 5. Synthesis of Precursor 8-ethoxytricyclo [5.2.1.02,6]decane-4-sulfanyl-1-(2,6,6-trimethylcyclohex-3-en-1-yl) butan-1-one (Compound of Formula (I) According to the Invention)

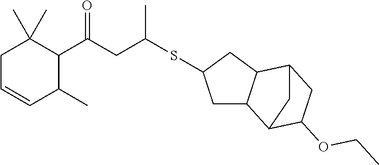 [SMILES: CCOC1CC2CC1C1CC(SC(C)CC(=O)C3C(C)C=CCC3(C)C)CC21]

In a 50 mL stirring apparatus with magnetic stirrer, reflux condenser, thermometer, dropping funnel and ice bath, 0.02 g of 1,1,3,3-tetramethylguanidine, 16.4 g of 8-ethoxytricyclo [5.2.1.02,6]decane-4-thiol and tetrahydrofuran (40 mL) were added under a nitrogen atmosphere at 0° C. At 0-5° C., 14.4 g of (E)-[1]-(2,6,6-trimethylcyclohex-3-en-1-yl) but-2-en-1-one were added dropwise within 1 hours. For post-reaction, stirring was continued for 6 hours at 0-5° C. The mixture was washed twice with saturated sodium bicarbonate solution at room temperature and then the solvents were evaporated on the rotary evaporator. 30.4 g crude of 8-ethoxytricyclo [5.2.1.02,6]decane-4-sulfanyl-1-(2,6,6-trimethylcyclohex-3-en-1-yl) butan-1-one were obtained.

Mass spectrometric analysis of product:

m/z: 133 (100), 67 (58), 123 (51), 91 (48), 81 (45), 239 (41), 79 (41), 107 (41), 41 (37), 69 (37), 29 (35), 105 (24), 103 (21), 55 (20), 43 (18), 122 (17), 77 (17).

### Synthesis Procedures of Thiols According to the Invention

### 1. 2-Methyl-1-[4-methyltetrahydropyran-2-yl]propane-1-thiol

2-Methyl-1-[4-methyltetrahydropyran-2-yl]propane-1-thiol was prepared in accordance to WO 2019/037862 A1:

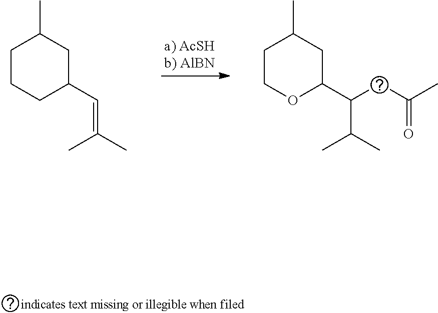

To 463 g 4-methyl-2-(2-methyl-prop-1-enyl)tetrahydropyran and 228 g thioacetic acid was added 2.46 g 2,2-azobis-(2-methyl-propionitril, AIBN) at 5-10° C. and stirred at room temperature for 30 min.

Afterwards, the mixture is slowly heated to 60° C. and stirred for 10 h at 60° C. until complete conversion. After cooling to room temperature, the product is obtained by distillation (2.0 mbar, 104-110° C.) to yield 527.69 g (76%) of 2-methyl-(1-4-methyltetrahdyropyran-2-yl) propyl-ethanethioate as a mixture of isomers.

EI-MS m/z (%): 231 (1, [M]⁺), 215 (1), 187 (1), 173 (1), 154 (4), 139 (4), 99 (100), 81 (15), 69 (12), 55 (12), 43 (42), 29 (4).

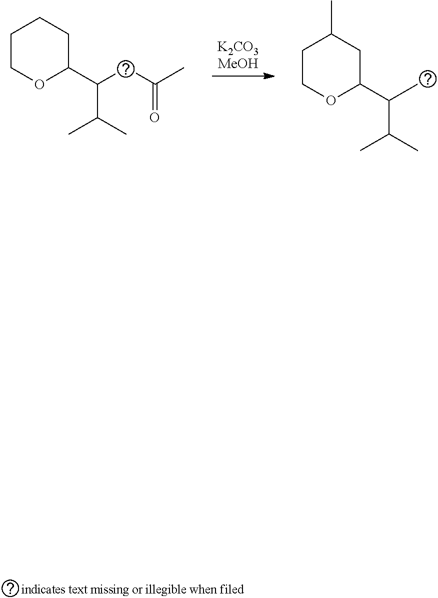

A solution of 501 g 2-methyl-(1-4-methyltetrahdyropyran-2-yl) propyl-ethanethioate in dry methanol (3.3 L) was treated with 300 g potassium carbonate und nitrogen and stirred over night until complete conversion. Afterwards, the mixture is acidified by 2 M hydrochloric acid (1 L) and 1 L H₂O and 1.5 L Et₂O are added. The organic phase is separated and the aqueous phase is extracted with 500 ml Et₂O. The combined organic phases are washed with brine (500 mL), dried over sodium sulfate and reduced in vacuum. The crude product is purified by distillation (3.6 mbar, 99-102° C.) to obtain 372 g (91%) 2-methyl-1-[4-methyltetrahydropyran-2-yl]propane-1-thiol as a mixture of isomers.

EI-MS m/z (%): 188 (1, [M]⁺), 170 (1), 139 (1), 111 (1), 99 (100), 81 (19), 69 (12), 55 (19), 43 (22), 29 (5).

### 2. 8-Ethoxytricyclo [5.2.1.02,6]decane-4-thiol

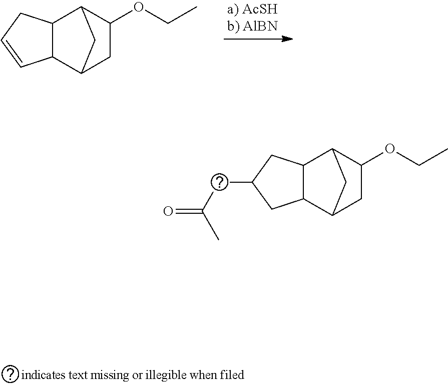 [SMILES: CCOC1CC2CC1C1CC=CC21 ; CCOC1CC2CC1C1CC(CC(C)=O)CC21]

To 583 g 8-methoxytricyclo [5.2.1.02,6]dec-3-ene and 279 g thioacetic acid was added 2.91 g 2,2-azobis-(2-methyl-propionitril, AIBN) at 5-10° C. and stirred at room temperature for 30 min. Afterwards, the mixture is slowly heated to 60° C. and stirred for 10 h at 60° C. until complete conversion. After cooling to room temperature, the product is obtained by distillation (2.0 mbar, 104-110° C.) to yield 773 g (91%) of (8-methoxytricyclo-4-[5.2.1.02,6]decanyl) ethanethioate as a mixture of isomers.

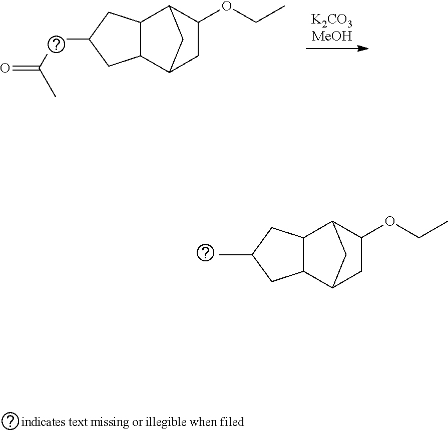

A solution of 538 g (8-methoxytricyclo-4-[5.2.1.02,6]decanyl) ethanethioate in dry methanol (3.3 L) was treated with 300 g potassium carbonate und nitrogen and stirred over night until complete conversion. The solvent is evaporated under reduced pressure and 400 mL MTBE is added. The organic phase is separated and the aqueous phase is extracted with 200 mL MTBE. The combined organic phases are washed with saturated sodium hydrogencarbonate solution, sodium sulfite solution and brine, dried over sodium sulfate and reduced under reduced pressure. The crude product is purified by distillation (3.6 mbar, 99-102° C.) to obtain 376 g (90%) 8-ethoxytricyclo [5.2.1.02,6]decane-4-thiol as a mixture of isomers.

Mass spectrometric analysis of product:

m/z: 166 (100), 91 (75), 92 (75), 133 (72), 79 (57), 132 (48), 105 (47), 117 (45), 93 (44), 67 (37), 106 (35), 212 (28), 77 (26), 29 (25), 41 (25), 66 (24), 137 (24).

### Products According to the Invention

### 1. Fabric Softener

TABLE 4
Fabric softener formulation according to the invention

| | | INCI Name / | |
|---|---|---|---|
| Ingredients | Supplier | description | (w/w) |
| Water, demin. | | Aqua | 86.60 |
| Rewoquat WE 18 | Evonik | Di-(tallow | 12.00 |
| | | carboxyethyl) | |
| | | hydroxyethyl | |
| | | methylammonium | |
| | | methosulfate | |
| Parmetol N 20 | Hoesch | | 0.10 |
| | Julius | | |
| Calcium Chloride 25% | | Calcium Chloride | 0.40 |
| solution | | | |
| 3-[2-methyl-1-(4- | | | 0.02 |
| methyltetrahydropyran- | | | |
| 2-yl)propyl]sulfanyl-1- | | | |
| (2,6,6- | | | |
| trimethylcyclohex-3- | | | |
| en-1-yl)butan-1-one | | | |
| Dipropylengylcol | Symrise | Dipropylengylcol, | 0.88 |
| | | mixture of isomers | |

### 2. Deodorant

TABLE 5
Deodorant formulation according to the invention

| Ingredients | Supplier | INCI Name/description | (w/w) |
|---|---|---|---|
| Propylenglycol 1-2 | Symrise | Propan-1,2-diol | 7.00 |
| Ethanol 96% | Symrise | Ethanol | 50.00 |
| Klucel HF | Uniqema | Hydrocxlpropyl cellulose | 0.80 |
| Water, demin | Symrise | Aqua | 39.65 |
| Deolite | Symrise | | 0.50 |
| Trilon B Liquid | BTC Europe | Na4-EDTA | 0.05 |
| | GmbH | | |
| Solubilizer | Symrise | PEG-40 hydrogenated | 1.00 |
| | | Castor oil, Trideceth-9, | |
| | | Propyleneglycol, Water | |
| 3-[2-methyl-1-(4- | | | 0.02 |
| methyltetrahydropyran- | | | |
| 2-yl)propyl]sulfanyl-1- | | | |
| (2,6,6- | | | |
| trimethylcyclohex-3- | | | |
| en-1-yl)butan-1-one | | | |
| Dipropylengylcol | Symrise | Dipropylengylcol, | 0.98 |
| | | mixture of isomers | |

### 3. Shampoo

TABLE 6
Shampoo formulation according to the invention

| | | INCI | |
|---|---|---|---|
| Ingredients | Supplier | Name/description | (w/w) |
| Water, demin | | Aqua | 76.84 |
| Plantacare PS 10 | Kraemer & | Sodium Lauryl | 20.00 |
| | | Sulphate, Lauryl | |
| | Martin | glucoside | |
| | GmbH | | |
| Sodium Chloride | Symrise | Sodium Chloride | 2.00 |
| Citric Acid E 330 | Symrise | Citric Acid | 0.164 |
| Preservative Complex | Symrise | | 0.50 |
| 3-[2-methyl-1-(4- | | | 0.08 |
| methyltetrahydropyran- | | | |
| 2-yl)propyl]sulfanyl-1- | | | |
| (2,6,6- | | | |
| trimethylcyclohex-3-en- | | | |
| 1-yl)butan-1-one | | | |
| Dipropylengylcol | Symrise | Dipropylengylcol, | 0.416 |
| | | mixture of isomers | |

### Sensorial Analyses

### 1. Headspace Gas Chromatographic/Mass Spectrometric Analysis

### Application of Precursor in Fabric Softener (Hand Wash):

1.0 g of a solution containing 1.0% compound of formula (I) according to the present invention in fabric softener (cf. Table 7 below for composition of the fabric softener) was added to 1 l H₂O in a 2 l beaker and mixed for a short period of time to form a homogeneous solution.

TABLE 7
Composition of the used fabric softener

| Ingredients | Supplier | INCI Name / description | (w/w) |
|---|---|---|---|
| Water, demin. | | Aqua | 87.50 |
| Rewoquat WE 18 | Evonik | Di-(tallow carboxyethyl) | 12.00 |
| | | hydroxyethyl | |
| | | methylammonium | |
| | | methosulfate | |
| Parmetol N 20 | Hoesch | Mixture of Bronopol and 2- | 0.10 |
| | Julius | octyl-2H-isothiazol-3-one | |
| Calcium Chloride 25 | | Calcium Chloride | 0.40 |
| % solution | | | |

Five pieces of cotton fabric (16×17 cm, 25 g each) were added and stirred using a magnetic stirrer for 30 min at room temperature. Afterwards, the pieces of cotton fabric were individually wrung (approx. 60 g each) and dried on a laundry rack in a self-made closed drying chamber (143 l) at a constant air exchange and temperature of 25° C. At defined points in time, volatiles were adsorbed on a Tenax® cartridge for 2 min at a continuous flow of 500 ml/min by means of a Gerstel gas sampling system.

Additional measurements were carried out in a self-made glass tube using two pieces of fabric after 1 hour of drying in the drying chamber. Two pieces of fabric were taken from the laundry rack and introduced into the glass tube (0.44 l) at room temperature. The volatiles were adsorbed at a continuous flow of air (500 ml/min) using a Gerstel gas sampling system, initially for 58 min on a waste Tenax® cartridge and then for 2 min on a fresh Tenax® cartridge. Sampling on a waste Tenax® cartridge was then continued until, at defined points in time, samplings on fresh Tenax® cartridges were carried out for 2 mins each after 1 h, 2 h, 3 h, 4 h, 5 h, 6 h, 7 h, 8 h, 24 h, 48 h, 72 h. These cartridges were then desorbed on a Gerstel Thermal Desorption Unit at 260° C. The volatiles were injected into an Agilent 6890 gas chromatograph with a DB-1 MS column (30 m, 0.25 mm, 0.25 μm) coupled to an Agilent mass spectrometer 5973 N. The volatiles were eluted with a flow of He at 1.4 ml/min using a temperature gradient moving from 50° C. (for 4 min) to 260° C. at 8° C./min.

Headspace concentrations were calculated by external standard calibrations using different concentrations of the corresponding fragrance materials (0.1, 0.25, 0.5, 1.0, 2.5, 5.0 ng/l) in EtOH. The corresponding fragrance materials are the fragrance materials that are released from a specific precursor/compound of formula (I), i.e. the corresponding α,β-unsaturated carbonyl compounds and thiol compounds. Each calibration solution (1 μl) was injected onto a clean, empty cartridge which was desorbed and analyzed using the same conditions as described above.

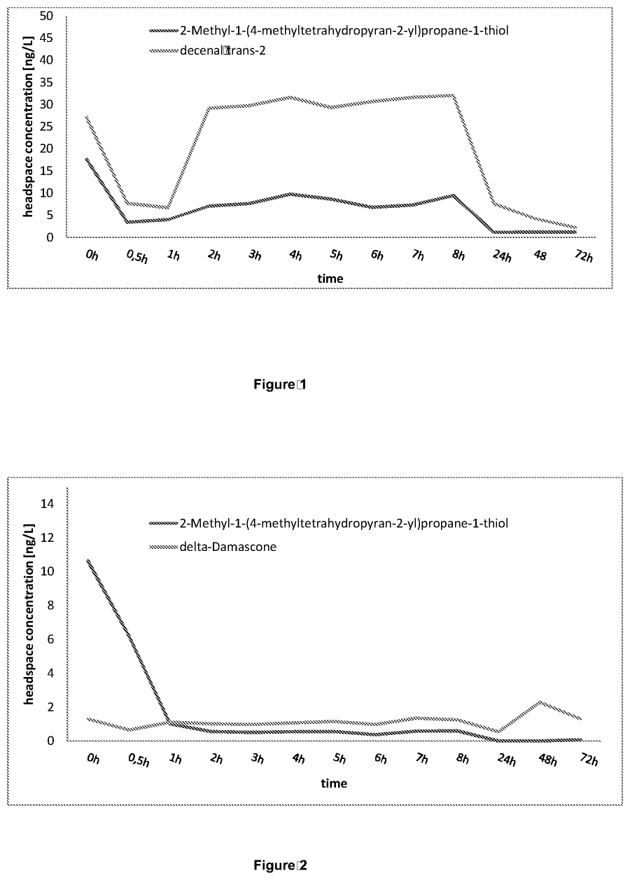

FIG. 1: Fragrance release curve for precursor 3-[2-methyl-1-(4-methyltetrahydropyran-2-yl)propyl]sulfanyldecanal (obtained according to procedure 2 as described above under synthesis procedures of precursors) as determined by procedure 1 as described above under sensorial analyses.

FIG. 2: Fragrance release curve for precursor 3-[2-methyl-1-(4-methyltetrahydropyran-2-yl)propyl]sulfanyl-1-(2,6,6-trimethylcyclohex-3-en-1-yl) butan-1-one (obtained according to procedure 1 as described above under synthesis procedures of precursors) as determined by procedure 1 as described above under sensorial analyses.

FIGS. 1 and 2 show the release of 2-methyl-1-(4-methyltetrahydropyran-2-yl)propanethiol and the respective α,β-unsaturated carbonyl compound from the compounds of formula (I) obtained according to procedures 1 and 2 as described above under synthesis procedures of precursors. The x axis of the Figures shows the period of time over which the measurement was conducted. On the y axis, the headspace concentrations (determined according to procedure 1 as described above under sensorial analyses) of the two components released from the compound of formula (I) are shown, respectively. From the Figures it can be seen that on the freshly washed/wet laundry a strong release of the thiol compound occurs, which is also sensory perceptible. On the other hand, a noticeable release of the α,β-unsaturated carbonyl compound occurs with a time delay on dried laundry after several hours (2-decenal: 2-48 h, delta-damascone: 24-72 h). These analytical results demonstrate the (controlled) decomposition of the compounds of formula (I) according to the invention with simultaneous release of the α,β-unsaturated aldehyde or ketone over time and are in line with the sensory evaluation of the washing tests (cf. below). The controlled decomposition of the compound of formula (I) advantageously leads to a long-lasting perception of the α,β-unsaturated aldehyde or ketone on the dried laundry. Additionally, the high impact thiol compound improves the overall scent of the freshly washed laundry without unwanted or unpleasant off notes.

For comparison, the experiment was repeated, however instead of using the compounds of formula (I) according to the invention, 1.0 g of a mixture of α,β-unsaturated carbonyl compound (hexenal) and thiol (2-methyl-1-(4-methyltetrahydropyran-2-yl)propane-1-thiol) was employed.

TABLE 8
Fragrance release for precursor 3-[2-methyl-1-(4-methyltetrahydropyran-
2-yl)propyl]sulfanylhexanal (compound of formula (I) according
to the invention) and a mixture of 34% hexenal and 66% 2-methyl-
1-(4-methyltetrahydropyran-2-yl)propane-1-thiol.

| | | | | c(Thiol) from | |
|---|---|---|---|---|---|
| | | c(Hexenal) from | | compound of | |
| | | compound of | c(Hexenal) | formula (I) | c(Thiol) |
| Time | | formula (I) [ng/L] | [ng/L] | [ng/L] | [ng/L] |
| 0 | h | 10.7 | 285.0 | 12.1 | 215.7 |
| 0.5 | h | 12.6 | 0.5 | 19.1 | 7.5 |
| 1 | h | 46.0 | 5.3 | 5.0 | 3.5 |
| 2 | h | 67.1 | 13.5 | 3.2 | 2.2 |
| 3 | h | 58.9 | 11.9 | 3.1 | 2.6 |
| 4 | h | 69.5 | 11.4 | 3.3 | 2.1 |
| 5 | h | 75.6 | 11.1 | 3.4 | 2.1 |
| 6 | h | 70.2 | 11.7 | 3.6 | 2.1 |
| 7 | h | 74.8 | 10.0 | 3.3 | 2.1 |
| 8 | h | 62.1 | 10.2 | 3.1 | 2.1 |
| 24 | h | 43.9 | 1 | 2.1 | 2.1 |
| 48 | h | 13.9 | — | 2.2 | — |
| 72 | h | 3.9 | 0.3 | 2.1 | 2.1 |

It can be seen that the fragrance release from the compound of formula (I) according to the invention is substantially different to the release from the mixture of α,β-unsaturated carbonyl compound and thiol, underlying that released fragrance materials are delivered from the partial degradation of the compound of formula (I) according to the invention over time. Unexpectedly, it is observed that the peak of thiol release is between 0-1 h, while the peak of release of hexenal is reached at 4-7 h, surprisingly revealing that the compounds of formula (I) according to the invention can deliver two pleasantly smelling fragrance materials at different points in time.

### 2. Washing Tests

A sensory evaluation of the compounds of formula (I) according to the invention (obtained according to procedures 1 and 2 as described above under synthesis procedures of precursors) after a washing application was carried out as described below and delivered the test results as shown in Table 9 below.
Application of the respective compound of formula (I) according to the invention in perfume oil in a realistic in-use test
Perfume oil dosage: 1% by weight in fabric softener (cf. Table 7 above for composition of the fabric softener)
Precursor dosage in perfume oil: 2% by weight
Cotton fabric cloths were washed at 30° C. in a washing machine using the short cycle program
The sensory evaluation was carried out by a panel of 5 persons. The intensities given in Table 9 on a scale of 0 (no odour) to 9 (very strong odour) are an average of three evaluations by three panelists, respectively. The odour descriptions given in Table 9 are an average of five evaluations by five panelists, respectively.

TABLE 9

| | | | | Perfume oil with 2% |
|---|---|---|---|---|
| | | | | 3-[2-methyl-1-(4- |
| | | | Perfume oil with 2% | methyltetrahydropyran- |
| | | | 3-[2-methyl-1-(4- | 2-yl)propyl]sulfanyl-1- |
| | | | methyltetrahydropyran- | (2,6,6-trimethylcyclohex- |
| | | Benchmark: | 2-yl)propyl]- | 3-en-1-yl)butan-1-one |
| | | Perfume oil with | sulfanyldecanal | (releases delta- |
| | | 2% dipropylene | (releases 2-decenal as | damascone as carbonyl |
| | | glycol | carbonyl compound) | compound) |
| Wet fabric - | | Floral, transparent, | Strongest effect, | Pleasant, soft, |
| fresh | | slight musky 5 | strong sulfuric-fruity | slight sulfuric-fruity |
| | | | odour 7-8 | note 5-6 |
| 4 | h | 3 | Same odour profile | Same odour profile |
| | | | as benchmark 3 | as benchmark 3 |
| 24 | h | 2 | Intense decenal | Intense damascone |
| | | | (for 24-48 h) | (for 24-120 h) |
| | | | 4 | 4-5 |
| 48 | h | 2 | 3 | 5 |
| 120 | h | 2 | 2 | 4 |

### 3. Fabric Softener

Perfumed fabric softener samples were prepared by mixing the appropriate amount of compound of formula (I) and dipropylene glycol (DPG) to the perfume oil and adding 1% of the resulting fragrance mixture to unperfumed fabric softener.

2 kg of cotton towels were washed in a European washing machine (Express 20 program, 20° C., 800 rpm) using 30 g of perfumed fabric softener (cf. Table 7 for composition of the fabric softener). The fabrics were lined dried over night and put on a table for evaluation after 24 h, 48 h, and 72 h. The fabrics were evaluated by 12 trained panelists. Perfume intensities are evaluated on a scale from no odor (0) to strongest imaginable (10). Panelists were also asked to choose which sample is perceived in higher intensity.

It is concluded that the fragrance perception is significantly stronger using compound of formula (I) in comparison to an equivalent amount of delta-damascone. To underline this, one piece of cotton towel was analyzed by headspace GC/MS according to procedure 1 as described above under sensorial analyses, revealing a significantly higher headspace concentration of delta-damascone of the cotton towel washed with perfume oil including compound of formula (I).

TABLE 10
Mean intensities

| | | | Perfume oil with 2% 3-[2- |
|---|---|---|---|
| | | | methyl-1-(4- |
| | | | methyltetrahydropyran-2- |
| | | | yl)propyl]sulfanyl-1- (2,6,6- |
| | | Perfume oil containing 1% | yl)butan-1-one (releases delta- |
| | | delta-damascone and 1% | damascone as carbonyl |
| | | DPG | compound) |
| 24 | h | 2.14 | 2.94 |
| 48 | h | 1.46 | 2.53 |
| 72 | h | 1.03 | 2.37 |
| 168 | h | 1.04 | 1.51 |

TABLE 11
Probability of strongest sample

| | | Perfume oil with 2% 3-[2- |
|---|---|---|
| | | methyl-1-(4- |
| | | methyltetrahydropyran-2- |
| | | yl)propyl]sulfanyl-1- (2,6,6- |
| | | trimethylcyclohex-3-en-1- |
| | Perfume oil containing 1% | yl)butan-1-one (releases delta- |
| | delta-damascone and 1% | damascone as carbonyl |
| | DPG | compound) |
| 24 h | 0.31 | 0.62 |
| 48 h | 0.08 | 0.92 |
| 72 h | 0.15 | 0.85 |
| 168 h | 0.36 | 0.64 |

TABLE 12
Composition of used perfume oil

| | |
|---|---|
| AGRUMEX LC | 40 |
| ALDEHYD C14 SOG | 5 |
| ALLYLCYCLOHEXYLPROPIONAT | 5 |
| ALLYLHEPTYLAT | 5 |
| AMBROXIDE | 5 |
| CITRONELLOL 950 | 10 |
| CYCLAMENALDEHYD | 10 |
| CYCLOGALBANAT ® | 1 |
| DECALACTON GAMMA | 3 |
| DIHYDROMYRCENOL | 300 |
| DIMETHYLBENZYLCARBINYLACETAT | 10 |
| DYNASCONE 10% DPG | 5 |
| ETHYLENBRASSYLAT | 45 |
| ETHYLVANILLIN 10% DPG | 5 |
| FLORAZON | 1 |
| FLOWERPOOL 0.1% DPG | 5 |
| FREESIOL / CORPS 119 | 50 |
| GLOBALIDE ® | 60 |
| HERBAFLORAT | 40 |
| HERBYLPROPIONAT | 25 |
| HEXENOL CIS-3 10% DPG | 35 |
| HEXYLZIMTALDEHYD ALPHA | 140 |
| KRAUSEMINZOEL 65% AMERIK. | 5 |
| MANZANATE 10% DPG | 5 |
| MELONAL | 15 |
| NEROLIN YARA YARA KRIST. | 10 |
| ORANGENOEL TERPENE | 40 |
| PHENIRAT ® | 20 |
| STYROLYLACETAT | 5 |
| TERPINEOL ALPHA | 10 |
| TIMBEROL ® | 10 |
| VANILLE EXTRAKT BOURBON 10X FAIR | 15 |
| 10% DPG | |
| VELOUTONE 10% DPG | 15 |
| VERTOCITRAL | 5 |
| YSAMBER ® K | 20 |

### Fragrance Release for Compound of Formula (I) 3-[2-methyl-1-(4-methyltetrahydropyran-2-yl)propyl]-sulfanyl-1-(2,6,6-trimethylcyclohex-3-en-1-yl)butan-1-one and a Mixture of 50% delta-damascone and 50% DPG

TABLE 13

| | c(damascone) from | |
|---|---|---|
| | compound of formula (I) | c(damascone) |
| Time | [ng/L] | [ng/L] |
| 24 h | 1.2 | 0.2 |
| 48 h | 4.8 | 0.2 |
| 72 h | 6.7 | 0.2 |
| 168 h | 0.3 | 0.2 |

### 4. Deodorant

An artificial skin model was prepared using synthetic skin (Vitro-Skin®, approx. 2.5 cm radius), fat, cellulose, and agar.

Perfumed deo samples were prepared by mixing the appropriate amount of compound of formula (I) and DPG to the perfume oil and adding 1% of the resulting fragrance mixture to unperfumed deo base.

100 mg of perfumed deo is added on artificial skin and evenly distributed for 15 s. Afterwards, the model is placed on a heating plate (43° C.) and evaluated after 0 h, 4 h, 24 h, and 48 h by 6 trained panelists. Perfume intensities are evaluated on a scale from no odor (0) to strongest imaginable (9).

It is concluded that the intensity of the fragrance is significantly stronger when applying a perfume oil including a compound of formula (I). Unexpectedly, it is revealed the perception of the individual precursor components (thiol, α,β-unsaturated carbonyl) occurs non-simultaneously, as the thiol component is perceived at 0 h, giving a richer fruity profile, while delta-damascone is perceived after 4-48 h without any perception of thiol notes.

TABLE 14
Mean intensities

| | | Perfume oil with 0.5% | Perfume oil with 1% | Perfume oil with 2% |
|---|---|---|---|---|
| | | 3-[2-methyl-1-(4- | 3-[2-methyl-1-(4- | 3-[2-methyl-1-(4- |
| | | methyltetrahydropyran-2- | methyltetrahydropyran-2- | methyltetrahydropyran-2- |
| | | yl)propyl]sulfanyl-1- | yl)propyl]sulfanyl-1- | yl)propyl]sulfanyl-1- |
| | | (2,6,6- | (2,6,6- | (2,6,6- |
| | | trimethylcyclohex- | trimethylcyclohex-3- | trimethylcyclohex- |
| | Perfume | 3-en-1-yl)butan-1- | en-1-yl)butan-1-one | 3-en-1-yl)butan-1- |
| | oil | one (releases delta- | (releases delta- | one (releases delta- |
| | containing | damascone as | damascone as | damascone as |
| | 2% DPG | carbonyl compound) | carbonyl compound) | carbonyl compound) |
| 0 h | 4.2 | 4.9 | 4.8 | 4.8 |
| 4 h | 3.2 | 4.3 | 4.6 | 4.7 |
| 24 h | 1.5 | 2.5 | 3.3 | 4 |
| 48 h | 1.6 | 2 | 3 | 3.3 |

Panelists were asked to describe the hedonic differences between the different samples.

TABLE 15
Sensory descriptions by panelists

| | | Perfume oil with 0.5% | Perfume oil with 1% | Perfume oil with 2% |
|---|---|---|---|---|
| | | 3-[2-methyl-1-(4- | 3-[2-methyl-1-(4- | 3-[2-methyl-1-(4- |
| | | methyltetrahydropyran-2- | methyltetrahydropyran-2- | methyltetrahydropyran-2- |
| | | yl)propyl]sulfanyl-1- | yl)propyl]sulfanyl-1- | yl)propyl]sulfanyl-1- |
| | Perfume | (2,6,6-trimethylcyclohex- | (2,6,6-trimethylcyclohex- | (2,6,6-trimethylcyclohex- |
| | oil | 3-en-1-yl)butan-1-one | 3-en-1-yl)butan-1-one | 3-en-1-yl)butan-1-one |
| | containing | (releases delta-damascone | (releases delta-damascone | (releases delta-damascone |
| | 2% DPG | as carbonyl compound) | as carbonyl compound) | as carbonyl compound) |
| 0 h | | More floral-creamy, | More floral, fruity, | More floral, fruity, |
| | | slight thiol note | slight thiol note | slight thiol note |
| | | (not unpleasant) | (not unpleasant | (not unpleasant |
| 4 h | | Stronger, more | Stronger, more | Stronger, more |
| | | floral, apple, | floral, apple, | floral, apple, |
| | | fruitier, | fruitier, | fruitier, |
| | | damascone | damascone | damascone |
| 24 h | | Fruity, apple, | Fruity, apple, | Fruity, apple, |
| | | damascone | damascone | damascone |
| 48 h | | Slight damascone, | Damascone, fruity, | Damascone, fruity, |
| | | slight plum | plum | dried plum |

TABLE 16
Composition of the used perfume oil

| | |
|---|---|
| AGRUMEX LC | 20 |
| ALDEHYD C 8 | 1.5 |
| ALDEHYD C11 UNDECYLENIC | 2 |
| 10% DPG | |
| ALDEHYD C12 LAURIN | 1 |
| ALDEHYD C18 SOG. | 3 |
| ALLYLCYCLOHEXYLPROPIONAT | 4 |
| AMAROCIT ® | 10 |
| BENZYLACETAT | 8 |
| BENZYLSALICYLAT | 15 |
| CITRAL 95 | 4 |
| CITRONELLOL 950 | 20 |
| CUMARIN | 3 |
| DECALACTON GAMMA | 13 |
| DIHYDROMYRCENYLACETAT | 23 |
| DIMETHYLBENZYLCARBINYLACETAT | 2 |
| DIPROPYLENGLYCOL | 183 |
| ETHYLENBRASSYLAT | 40 |
| ETHYLLINALOOL | 8 |
| EVERNYL 10% DPG | 5 |
| Ecomusk R | 10 |
| FLOROPAL | 3 |
| FLOROSA BM/PYRANOL | 22 |
| GERANIOL SUPRA | 15 |
| GERANYLACETAT PUR | 5 |
| GLOBALIDE ® | 15 |
| GRAPEFRUITOEL TERPENE | 80 |
| HELIONAL | 8 |
| HELIOTROPIN/PIPERONAL | 3 |
| HEXENOL CIS-3 | 0.5 |
| HEXENYLACETAT CIS-3 | 1 |
| HEXENYLSALICYLAT CIS-3 | 4 |
| HEXYLSALICYLAT | 25 |
| HEXYLZIMTALDEHYD ALPHA | 45 |
| INDOFLOR ® KRIST. 10% DPG | 2 |
| IONON ALPHA | 2 |
| ISO E SUPER | 35 |
| ISOPROPYLMETHYLBUTYRAT-2 | 1 |
| LILYBELLE ® 10% DPG | 3 |
| LINALOOL | 20 |
| LINALYLACETAT | 15 |
| MAJANTOL ® | 2 |
| METHYL IONONE 70 | 25 |
| ORANGENOEL ITAL. SUESS ENTF. | 60 |
| PETITGRAINOEL PARAG. BOLEADOR | 1 |
| PHENYLETHYLALKOHOL | 30 |
| PHENYLETHYLPHENYLACETAT | 3 |
| ROSAPHEN ® | 8 |
| STYROLYLACETAT | 12 |
| SYMFRESH ® NX | 150 |
| TERPINEOL REIN | 7 |
| THIOCINEOL 100P 1% DPG | 4 |
| VERTACETAL ® COEUR | 2 |
| VERTOCITRAL 10% DPG | 6 |

TABLE 17
Composition of the used deodorant

| Ingredients | Supplier | INCI Name/description | (w/w) |
|---|---|---|---|
| Propylenglycol 1-2 | Symrise | Propan-1,2-diol | 7.00 |
| Ethanol 96% | Symrise | Ethanol | 50.00 |
| Klucel HF | Uniqema | Hydrocxlpropyl cellulose | 0.80 |
| Water, demin | Symrise | Aqua | 39.65 |
| Deolite | Symrise | | 0.50 |
| Trilon B Liquid | BTC Europe | Na4-EDTA | 0.05 |
| | GmbH | | |
| Solubilizer | Symrise | PEG-40 hydrogenated | 1.00 |
| | | Castor oil, Trideceth-9, | |
| | | Propyleneglycol, Water | |

### 5. Shampoo

Perfumed shampoo samples were prepared by mixing the appropriate amount of compound of formula (I) and DPG to the perfume oil and adding 1% of the resulting fragrance mixture to unperfumed shampoo base.

One slightly wet hair swatch (2 g Caucasian hair) is treated with 1 g of perfumed shampoo and carefully washed for 15 s. Afterwards, the swatch is left to stand for 30 s and rinsed with warm water (30° C.) for 15 s. Excess water is removed by passing the swatch between the fingers.

The hair swatches were lined dried and evaluated after 0 h, 4 h, and 24 h by 5 trained panelists. Perfume intensities are evaluated on a scale from no odor (0) to strongest imaginable (9).

TABLE 18
Mean intensities

| | | Perfume oil with 1% | Perfume oil with 2% |
|---|---|---|---|
| | | 3-[2-methyl-1-(4- | 3-[2-methyl-1-(4- |
| | | methyltetrahydropyran- | methyltetrahydropyran- |
| | | 2-yl)propyl]sulfanyl-1- | 2-yl)propyl]sulfanyl-1- |
| | | (2,6,6-trimethylcyclohex- | (2,6,6-trimethylcyclohex- |
| | | 3-en-1-yl)butan-1-one | 3-en-1-yl)butan-1-one |
| | Perfume oil | (releases delta- | (releases delta- |
| | containing | damascone as | damascone as |
| | 2% DPG | carbonyl compound) | carbonyl compound) |
| 0 h | 5 | 5 | 4.6 |
| 4 h | 3.6 | 4.2 | 4 |
| 24 h | 0.7 | 1.3 | 2 |

Panelists were asked to describe the hedonic differences between the different samples.

TABLE 19
Sensory descriptions by panelists

| | | Perfume oil with 1% | Perfume oil with 2% |
|---|---|---|---|
| | | 3-[2-methyl-1-(4- | 3-[2-methyl-1-(4- |
| | | methyltetrahydropyran- | methyltetrahydropyran- |
| | | 2-yl)propyl]sulfanyl-1- | 2-yl)propyl]sulfanyl-1- |
| | | (2,6,6-trimethylcyclohex- | (2,6,6-trimethylcyclohex- |
| | | 3-en-1-yl)butan-1-one | 3-en-1-yl)butan-1-one |
| | Perfume oil | (releases delta- | (releases delta- |
| | containing | damascone as | damascone as |
| | 2% DPG | carbonyl compound) | carbonyl compound) |
| 0 h | | More creamy-caring | Slightly more fougère |
| 4 h | | Fruity, apple | Fruity, damascone |
| 24 h | | Fruity, apple | Fruity, fresh, |
| | | | creamier, damascone |

TABLE 20
Composition of the used perfume oil

| | |
|---|---|
| AGRUMEX LC | 100 |
| ALLYLAMYLGLYCOLAT | 10 |
| BEIFUSSOEL | 10 |
| BRAHMANOL ® | 25 |
| DIHYDROMYRCENOL | 200 |
| DIPROPYLENGLYCOL | 194 |
| ETHYLMALTOL 10% DPG | 12 |
| EVERNYL 10% DPG | 70 |
| FICHTENNADELOEL SIB. TYP | 15 |
| FRAMBINON ® 10% DPG | 10 |
| GERANIUMOEL MADAG. | 5 |
| HEXYLSALICYLAT | 119 |
| ISOBORNYLACETAT | 20 |
| LAVANDIN ESPIEUR CENSO | 60 |
| PATCHOULIOEL ENTF. | 50 |

TABLE 21
Composition of the used shampoo

| | | INCI | |
|---|---|---|---|
| Ingredients | Supplier | Name/description | (w/w) |
| Water, demin | Symrise | Aqua | 76.84 |
| Plantacare PS 10 | Kraemer & | Sodium Lauryl | 20.00 |
| | Martin GmbH | Sulphate, Lauryl | |
| | | glucoside | |
| Sodium Chloride | Symrise | Sodium Chloride | 2.00 |
| Citric Acid E 330 | Symrise | Citric Acid | 1.64 |
| Preservative | Symrise | | 0.50 |
| Complex | | | |

### 6. Deodorant

An artificial skin model was prepared using synthetic skin (Vitro-Skin®, approx. 2.5 cm radius), fat, cellulose, and agar.

Perfumed deo samples were prepared by mixing the appropriate amount of precursor to the perfume oil and adding 1% of the resulting fragrance mixture to unperfumed deo base. 100 mg of perfumed deo is added on artificial skin and evenly distributed for 15 s. Afterwards, the model is placed on a heating plate (43° C.) and evaluated after 0 h, 4 h, 24 h, and 48 h by 12 trained panelists. Perfume intensities are evaluated on a scale from no odor (0) to strongest imaginable (9).

It is detected that the perfume oil containing compound of formula (I) according to the invention is perceived stronger compared to perfume oil containing precursor of the prior art (WO 2015/032885 A1) at all stages of evaluation, showing a clear benefit of the compounds of formula (I) according to the invention. Surprisingly, both the hedonic profile and the perceived intensity of the fragrance after 0 h are positively influenced by introduction of a pleasantly smelling thiol component, while the superior and long-lasting performance at 6-24 h is likely due to the lower evaporation profile/higher substantivity of the compound of formula (I) according to the invention.

TABLE 22
Probability of strongest sample

| | | Perfume oil with 1% |
|---|---|---|
| | | 3-[2-methyl-1-(4- |
| | Perfume oil with 0.8% | methyltetrahydropyran- |
| | pentanethiol damascone | 2-yl)propyl]sulfanyl-1- |
| | (releases delta-damascone | (2,6,6-trimethylcyclohex- |
| | as carbonyl compound; | 3-en-1-yl)butan-1-one |
| | cf. I-C5 of WO | (releases delta-damascone |
| | 2015/032885 A1) | as carbonyl compound) |
| 0 h | 0.19 | 0.81 |
| 4 h | 0.27 | 0.73 |
| 6 h | 0.31 | 0.69 |
| 24 h | 0.20 | 0.80 |

TABLE 23
Mean intensities

| | | Perfume oil with 1% |
|---|---|---|
| | | 3-[2-methyl-1-(4- |
| | Perfume oil with 0.8% | methyltetrahydropyran- |
| | pentanethiol damascone | 2-yl)propyl]sulfanyl-1- |
| | (releases delta-damascone | (2,6,6-trimethylcyclohex- |
| | as carbonyl compound; | 3-en-1-yl)butan-1-one |
| | cf. I-C5 of WO | (releases delta-damascone |
| | 2015/032885 A1) | as carbonyl compound) |
| 0 h | 5.1 | 5.9 |
| 4 h | 3.8 | 4.7 |
| 6 h | 3.2 | 3.8 |
| 24 h | 2.6 | 3.1 |

TABLE 24
Probability of strongest sample

| | | Perfume oil with 1% |
|---|---|---|
| | | 3-[2-methyl-1-(4- |
| | Perfume oil with 1% | methyltetrahydropyran- |
| | 3-(dodecylthio)-1-(2,6,6- | 2-yl)propyl]sulfanyl-1- |
| | trimethyl-3-cyclohexen-1- | (2,6,6-trimethylcyclohex- |
| | yl)-1-butanone (releases | 3-en-1-yl)butan-1-one |
| | delta-damascone as | (releases delta-damascone |
| | carbonyl compound) | as carbonyl compound) |
| 0 h | 0.46 | 0.54 |
| 4 h | 0.46 | 0.54 |
| 6 h | 0.45 | 0.55 |
| 24 h | 0.29 | 0.71 |
| 48 h | 0.27 | 0.73 |

TABLE 25
Mean intensities

| | | Perfume oil with 1% |
|---|---|---|
| | | 3-[2-methyl-1-(4- |
| | Perfume oil with 1% | methyltetrahydropyran- |
| | 3-(dodecylthio)-1-(2,6,6- | 2-yl)propyl]sulfanyl-1- |
| | trimethyl-3-cyclohexen-1- | (2,6,6-trimethylcyclohex- |
| | yl)-1-butanone (releases | 3-en-1-yl)butan-1-one |
| | delta-damascone as | (releases delta-damascone |
| | carbonyl compound) | as carbonyl compound) |
| 0 h | 5.2 | 5.3 |
| 4 h | 4.1 | 4.2 |
| 6 h | 3.5 | 4.0 |
| 24 h | 3 | 3.3 |
| 48 h | 2.1 | 2.8 |

TABLE 26
Composition of used perfume oil

| | |
|---|---|
| AGRUMEX LC | 20 |
| ALDEHYD C 8 | 1.5 |
| ALDEHYD C11 UNDECYLENIC | 2 |
| 10% DPG | |
| ALDEHYD C12 LAURIN | 1 |
| ALDEHYD C18 SOG. | 3 |
| ALLYLCYCLOHEXYLPROPIONAT | 4 |
| AMAROCIT ® | 10 |
| BENZYLACETAT | 8 |
| BENZYLSALICYLAT | 15 |
| CITRAL 95 | 4 |
| CITRONELLOL 950 | 20 |
| CUMARIN | 3 |
| DECALACTON GAMMA | 13 |
| DIHYDROMYRCENYLACETAT | 23 |
| DIMETHYLBENZYLCARBINYLACETAT | 2 |
| DIPROPYLENGLYCOL | 183 |
| ETHYLENBRASSYLAT | 40 |
| ETHYLLINALOOL | 8 |
| EVERNYL 10% DPG | 5 |
| Ecomusk R | 10 |
| FLOROPAL | 3 |
| FLOROSA BM/PYRANOL | 22 |
| GERANIOL SUPRA | 15 |
| GERANYLACETAT PUR | 5 |
| GLOBALIDE ® | 15 |
| GRAPEFRUITOEL TERPENE | 80 |
| HELIONAL | 8 |
| HELIOTROPIN/PIPERONAL | 3 |
| HEXENOL CIS-3 | 0.5 |
| HEXENYLACETAT CIS-3 | 1 |
| HEXENYLSALICYLAT CIS-3 | 4 |
| HEXYLSALICYLAT | 25 |
| HEXYLZIMTALDEHYD ALPHA | 45 |
| INDOFLOR ® KRIST. 10% DPG | 2 |
| IONON ALPHA | 2 |
| ISO E SUPER | 35 |
| ISOPROPYLMETHYLBUTYRAT-2 | 1 |
| LILYBELLE ® 10% DPG | 3 |
| LINALOOL | 20 |
| LINALYLACETAT | 15 |
| MAJANTOL ® | 2 |
| METHYL IONONE 70 | 25 |
| ORANGENOEL ITAL. SUESS ENTF. | 60 |
| PETITGRAINOEL PARAG. BOLEADOR | 1 |
| PHENYLETHYLALKOHOL | 30 |
| PHENYLETHYLPHENYLACETAT | 3 |
| ROSAPHEN ® | 8 |
| STYROLYLACETAT | 12 |
| SYMFRESH ® NX | 150 |
| TERPINEOL REIN | 7 |
| THIOCINEOL 100P 1% DPG | 4 |
| VERTACETAL ® COEUR | 2 |
| VERTOCITRAL 10% DPG | 6 |

### 7. Fabric Softener

Perfumed fabric softener samples were prepared by mixing the appropriate amount of precursor to the perfume oil and adding 1% of the resulting fragrance mixture to unperfumed fabric softener.

2 kg of cotton towels were washed in a European washing machine (Express 20 program, 20° C., 800 rpm) using 30 g of perfumed fabric softener (cf. Table 7 for composition of the fabric softener). The fabrics were lined dried over night and put on a table for evaluation after 0 h, 24 h, 48 h, 72 h, and 144 h. The fabrics were evaluated by 15 trained panelists. Perfume intensities are evaluated on a scale from no odor (0) to strongest imaginable (10). Panelists were also asked to choose which sample is perceived in higher intensity.

It is detected that the perfume oil containing compound of formula (I) according to the invention is perceived stronger compared to precursor of the prior art (WO 2015/032885 A1) at all stages of evaluation, showing a clear benefit of the present system. Surprisingly, both the hedonic profile and the perceived intensity of the fragrance after 0 h are positively influenced by introduction of a pleasantly smelling thiol component, while the superior, long-lasting performance at 24-48 h is likely due to the lower evaporation profile/higher substantivity of the compound of formula (I) according to the invention.

TABLE 27
Probability of strongest sample

| | | Perfume oil with 1% |
|---|---|---|
| | | 3-[2-methyl-1-(4- |
| | Perfume oil with 0.8% | methyltetrahydropyran- |
| | pentanethiol damascone | 2-yl)propyl]sulfanyl-1- |
| | (releases delta-damascone | (2,6,6-trimethylcyclohex- |
| | as carbonyl compound; | 3-en-1-yl)butan-1-one |
| | cf. I-C5 of WO | (releases delta-damascone |
| | 2015/032885 A1) | as carbonyl compound) |
| 0 h | 0.46 | 0.54 |
| 24 h | 0.07 | 0.93 |
| 48 h | 0.12 | 0.88 |
| 72 h | 0.45 | 0.55 |
| 144 h | 0.57 | 0.43 |

TABLE 28
Mean intensities

| | | Perfume oil with 1% |
|---|---|---|
| | | 3-[2-methyl-1-(4- |
| | Perfume oil with 0.8% | methyltetrahydropyran- |
| | pentanethiol damascone | 2-yl)propyl]sulfanyl-1- |
| | (releases delta-damascone | (2,6,6-trimethylcyclohex- |
| | as carbonyl compound; | 3-en-1-yl)butan-1-one |
| | cf. I-C5 of WO | (releases delta-damascone |
| | 2015/032885 A1) | as carbonyl compound) |
| 1 h | 4.70 | 5.00 |
| 24 h | 2.1 | 3.2 |
| 48 h | 1.8 | 2.6 |
| 72 h | 1.7 | 1.7 |
| 144 h | 1.5 | 1.7 |

TABLE 29
Composition of the used perfume oil

| | |
|---|---|
| AGRUMEX LC | 20 |
| ALDEHYD C14 SOG. | 10 |
| ALLYLPHENOXYACETAT | 40 |
| BENZYLACETAT | 60 |
| BERGAMOT SYNTH. AFRIC. | 5 |
| W/O MYRCENE | |
| CITRONELLOL 950 | 135 |
| CYCLAMENALDEHYD | 100 |
| DAMASCON ALPHA 10% DPG | 5 |
| DIHYDROMYRCENOL | 25 |
| DIMETHYLBENZYLCARBINYLACETAT | 30 |
| DIPROPYLENGLYCOL | 10 |
| FRAMBINON ® 10% DPG | 10 |
| HEDION | 10 |
| HERBAFLORAT | 100 |
| HEXYLZIMTALDEHYD ALPHA | 35 |
| ISOBORNYLACETAT | 25 |
| ISOMUSCONE ® | 25 |
| LAVANDIN GROSSO RCO | 35 |
| LINALOOL | 40 |
| LINALYLACETAT | 10 |
| MANZANATE | 5 |
| MENTHANYLACETAT | 40 |
| METHYL IONONE 70 | 25 |
| MUSCENONE | 40 |
| PHENYLACETALDEHYD | 5 |
| DIMETHYLACETAL | |
| PHENYLETHYLACETAT | 5 |
| PHENYLPROPYLALKOHOL | 5 |
| ROSACETAT | 50 |
| TERPINEOL REIN | 65 |
| TIMBERSILK | 30 |

1-15. (canceled)

16. A compound of formula (I) 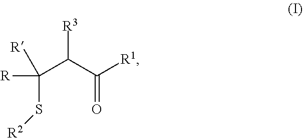
(i) wherein R is selected from the group consisting of hydrogen and C₁ to C₁₀ linear or cyclic alkyl, alkenyl or alkadienyl residues,
R′ is selected from the group consisting of hydrogen and C₁ to C₁₀ linear or cyclic alkyl, alkenyl or alkadienyl residues,
R¹ is selected from the group consisting of hydrogen and C₁ to C₁₀ linear or cyclic alkyl, alkenyl or alkadienyl residues,
R² is selected from the group consisting of C₄ to C₁₅ linear, cyclic or branched alkyl residues, additionally comprising one, two or more functional groups selected from the group consisting of ether, alcohol, and ketone, and
R³ is selected from the group consisting of hydrogen and C₁ to C₁₀ linear or cyclic alkyl, alkenyl or alkadienyl residues, or
(ii) wherein R¹, R² and R³ are independently selected from the groups as defined in (i), respectively, and R and R¹ are bonded together to form a saturated or unsaturated ring system comprising 5 to 10 carbon atoms, or
(iii) wherein R¹, R¹ and R² are independently selected from the groups as defined in (i), respectively, and R and R³ are bonded together to form a saturated or unsaturated, mono or bicyclic, ring system comprising 5 to 10 carbon atoms and optionally 1 or 2 ether functional groups.

17. The compound of claim 16, wherein R is selected from the group consisting of

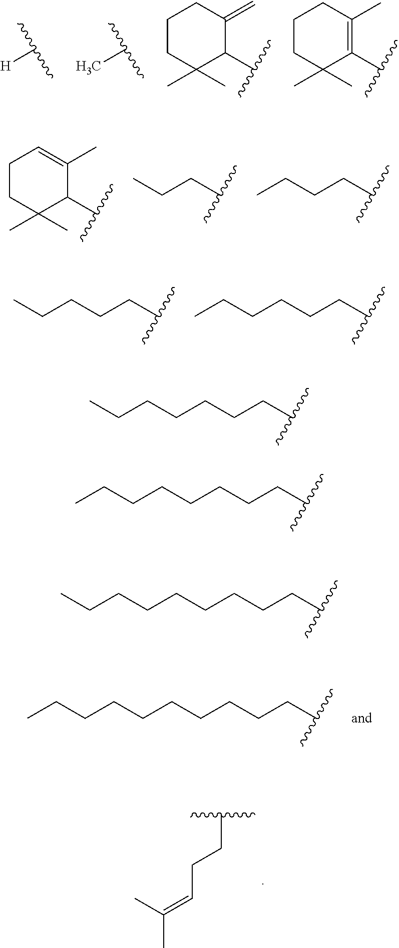

18. The compound of claim 16, wherein R′ is selected from the group consisting of

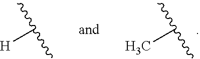

19. The compound of claim 16, wherein R¹ is selected from the group consisting of

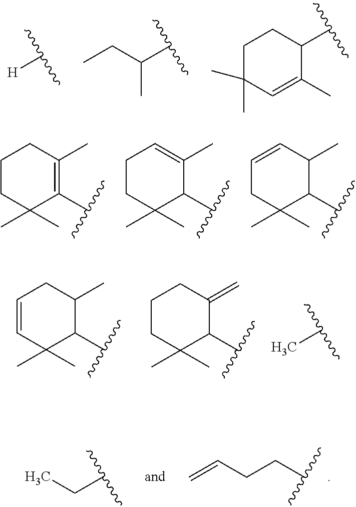

20. The compound of claim 16, wherein R² is selected from the group consisting of 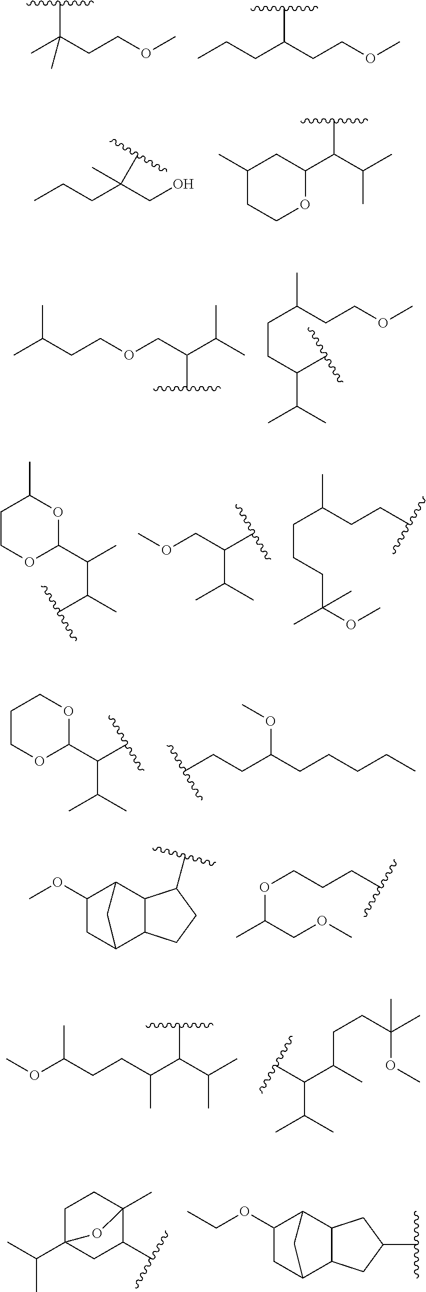 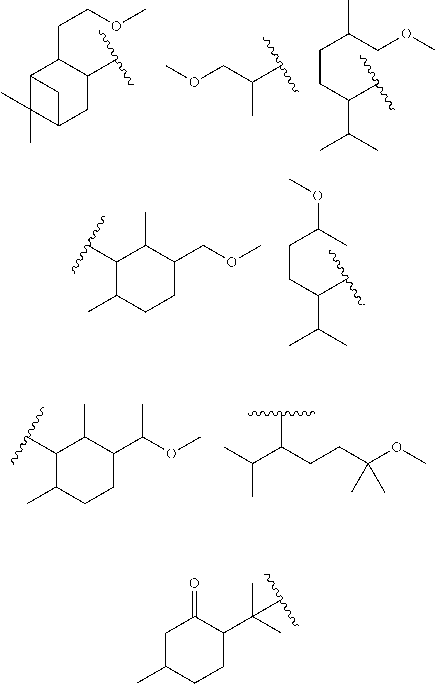
and

21. The compound of claim 16 obtained or obtainable by a [1,4]-addition reaction between:
an α,β-unsaturated aldehyde or ketone selected from the group consisting of 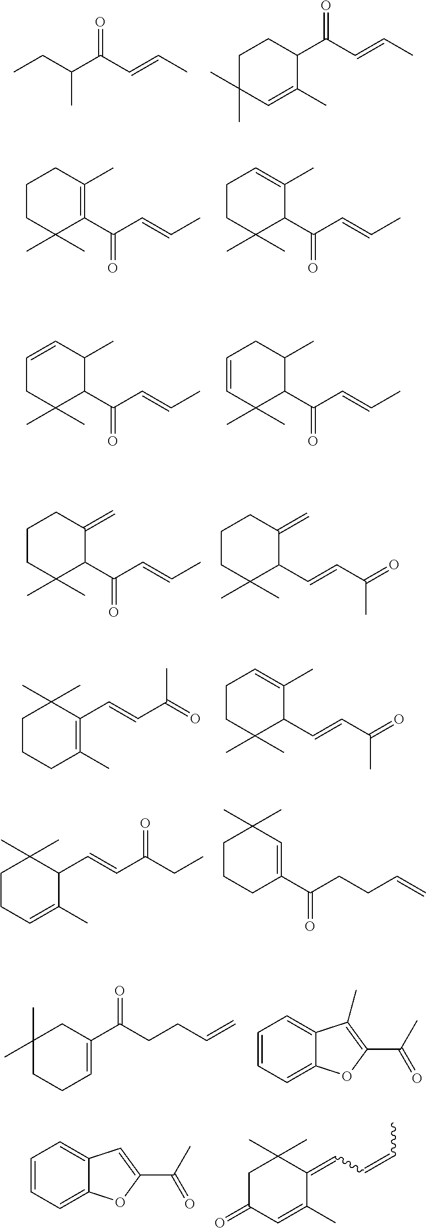 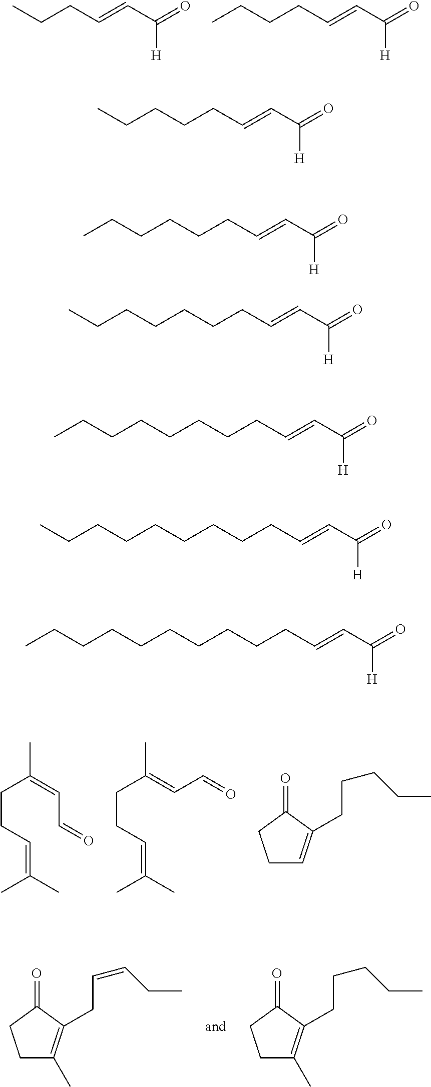
and
a thiol selected from the group consisting of

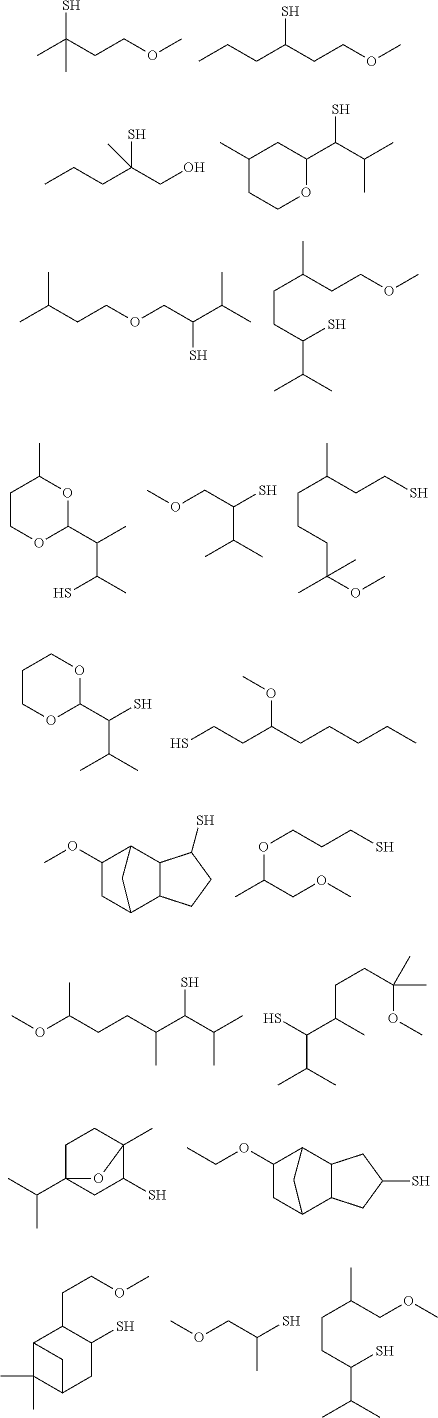

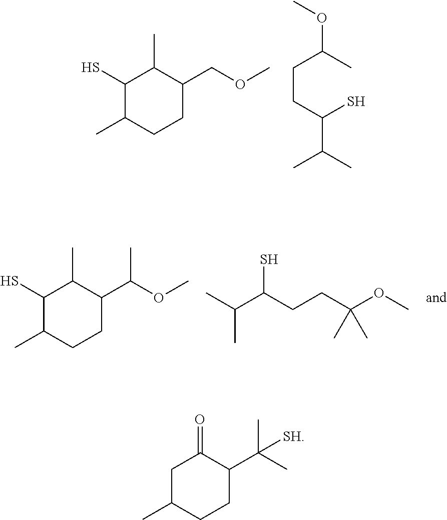

22. The compound of claim 16, wherein the compound of formula (I) is selected from the group consisting of

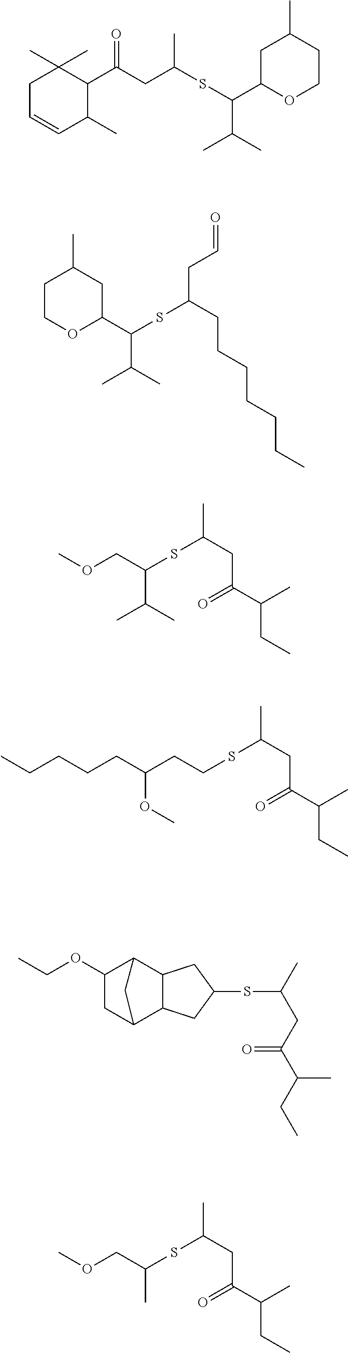

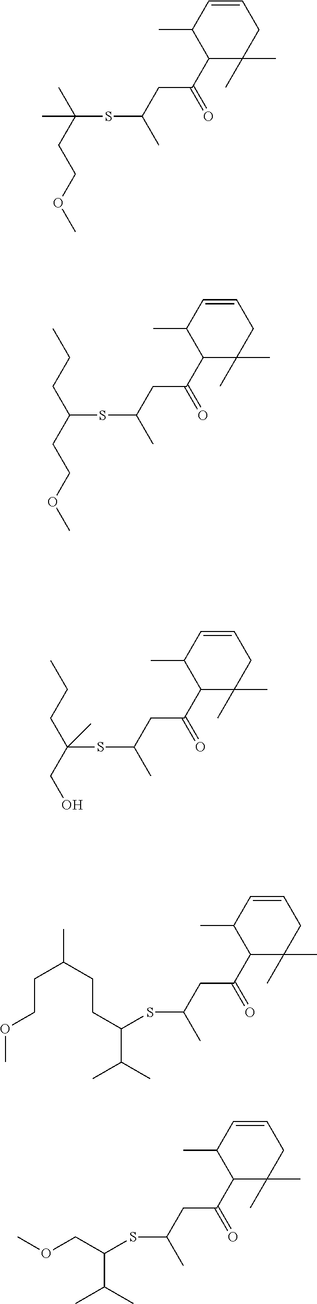

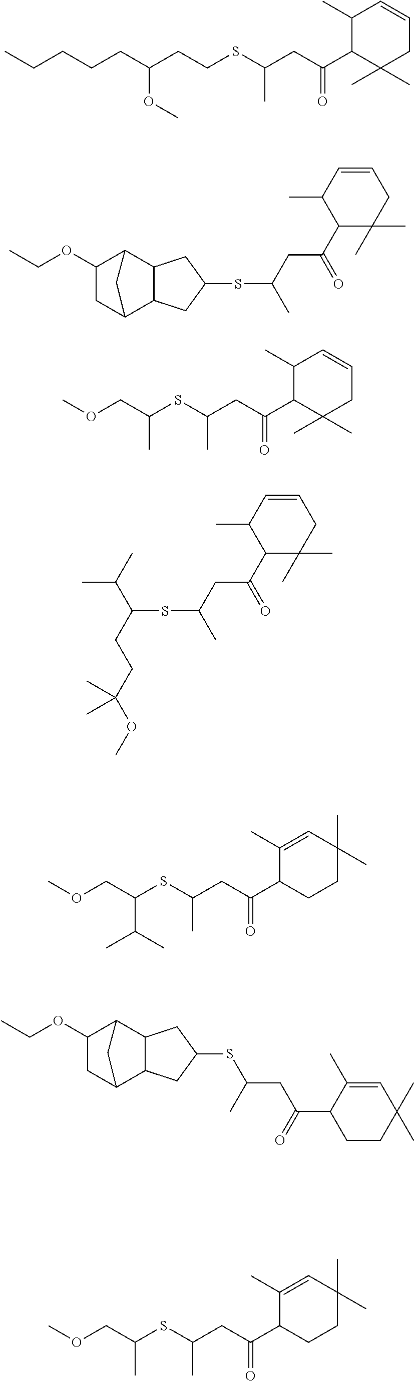

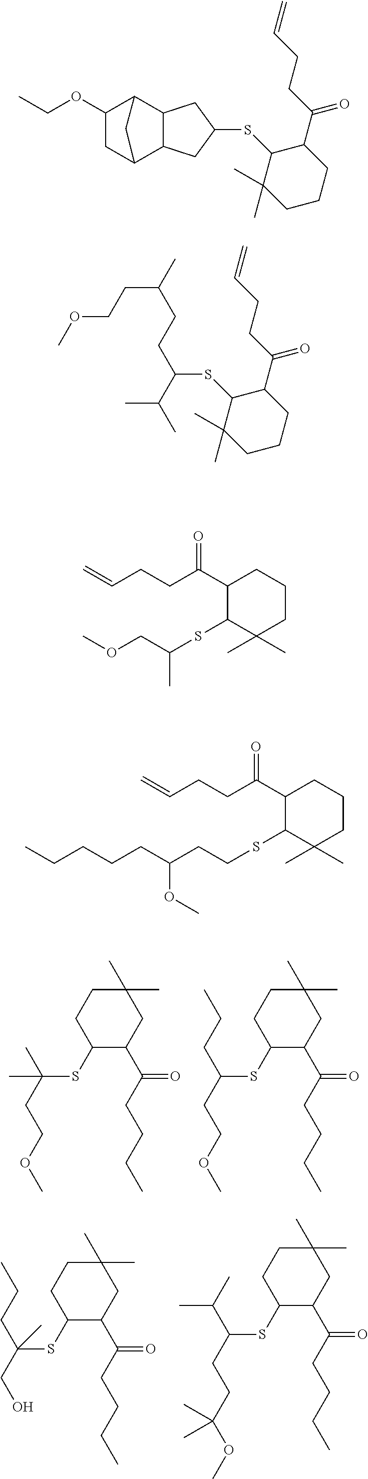

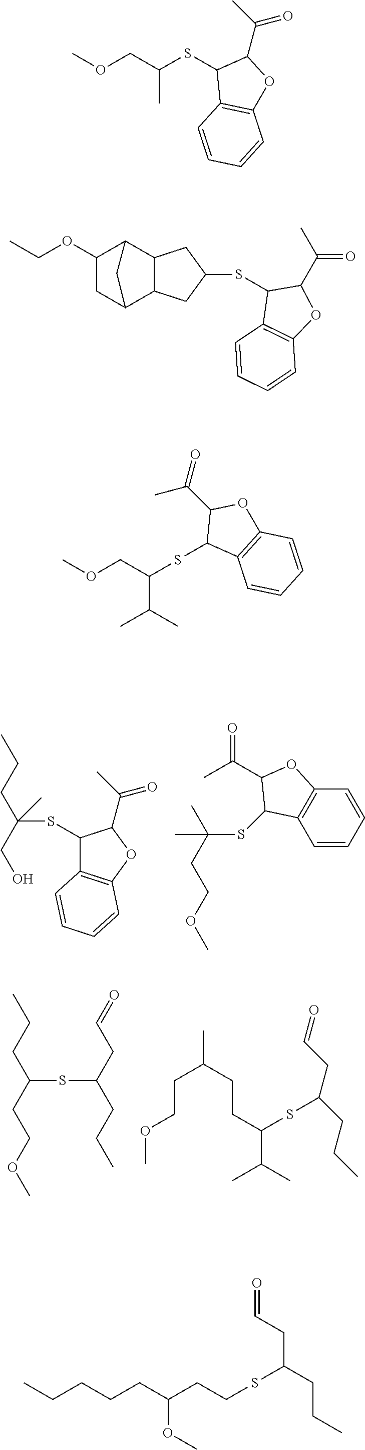

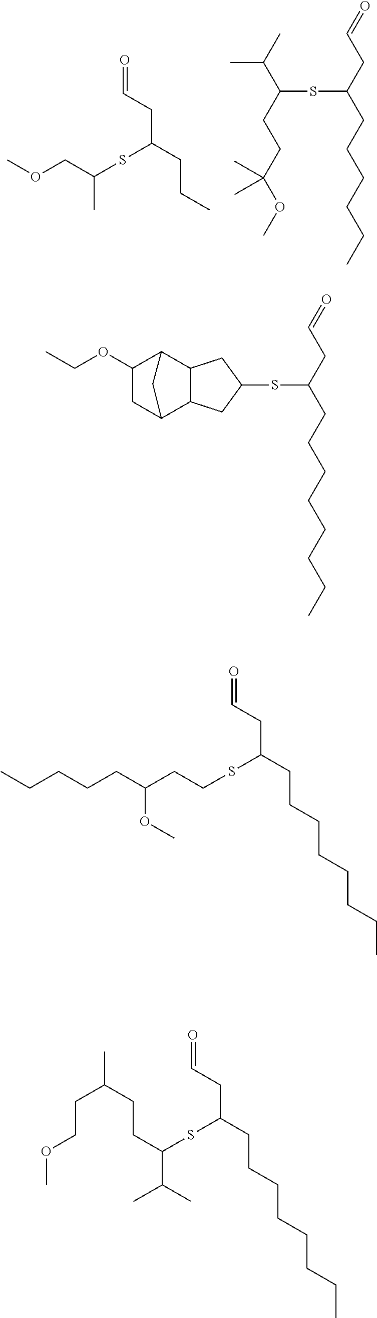

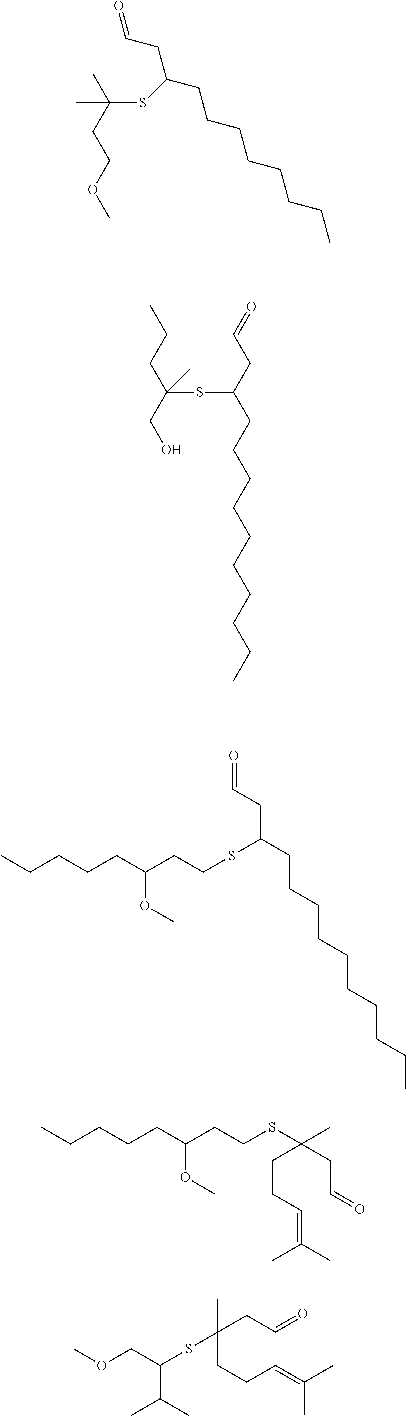

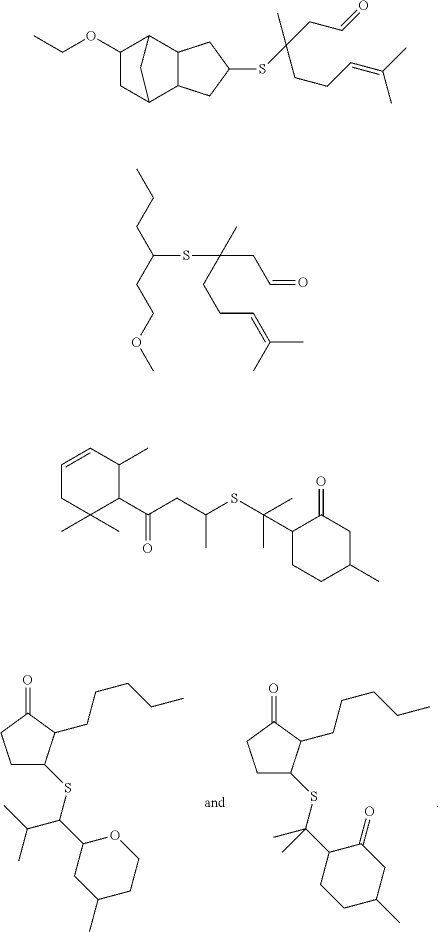 [SMILES: CCOC1CC2CC1C1CC(SC(C)(CC=O)CCC=C(C)C)CC21 ; CCCC(CCOC)SC(C)(CC=O)CCC=C(C)C ; CC1CCC(C(C)(C)SC(C)CC(=O)C2C(C)C=CCC2(C)C)C(=O)C1 ; CCCCCC1C(=O)CCC1SC(C(C)C)C1CC(C)CCO1 ; CCCCCC1C(=O)CCC1SC(C)(C)C1CCC(C)CC1=O]

23. A fragrance mixture comprising:
(i) one or more compounds of claim 16, and
(ii) one or more further fragrance substances.

24. A method for producing a fragrance substance mixture of claim 23 comprising mixing of one or more compounds of claim 1 with one or more further fragrance substances.

25. A perfumed product comprising one or more compounds of claim 16 in a sensorially effective amount.

26. The perfumed product of claim 25, wherein the product is selected from perfume extracts, eau de parfums, eau de toilettes, aftershaves, eau de colognes, pre-shave products, splash colognes, perfumed refreshing cloths, acidic, alkaline and neutral detergents, textile fresheners, ironing aids, liquid detergents, powdered detergents, laundry pre-treatment agents, fabric softeners, washing soaps, washing tablets, disinfectants, surface disinfectants, air fresheners, aerosol sprays, waxes and polishes, personal care products, hand creams and lotions, foot creams and lotions, depilatory creams and lotions, aftershave creams and lotions, tanning creams and lotions, hair care products, deodorants and antiperspirants, decorative cosmetic products, candles, lamp oils, incense sticks, insecticides, repellents, and fuels.

27. A method for producing a perfumed product of claim 25 comprising:
(i) providing one or more compounds of formula (I);
(ii) providing one or more further components of the perfumed product to be produced, and
(iii) contacting or mixing the further components with a sensorially effective amount of the one or more compounds of formula (I).

28. A method for release of two or more different pleasant smelling fragrance substances comprising:
(a) providing a compound of claim 16;
(b) optionally, applying or introducing the compound to hair, skin, textile fibres, a surface to be perfumed, or into ambient air to be perfumed; and
(c) releasing two or more different pleasantly smelling fragrance substances from the compound.

29. A thiol selected from the group consisting of

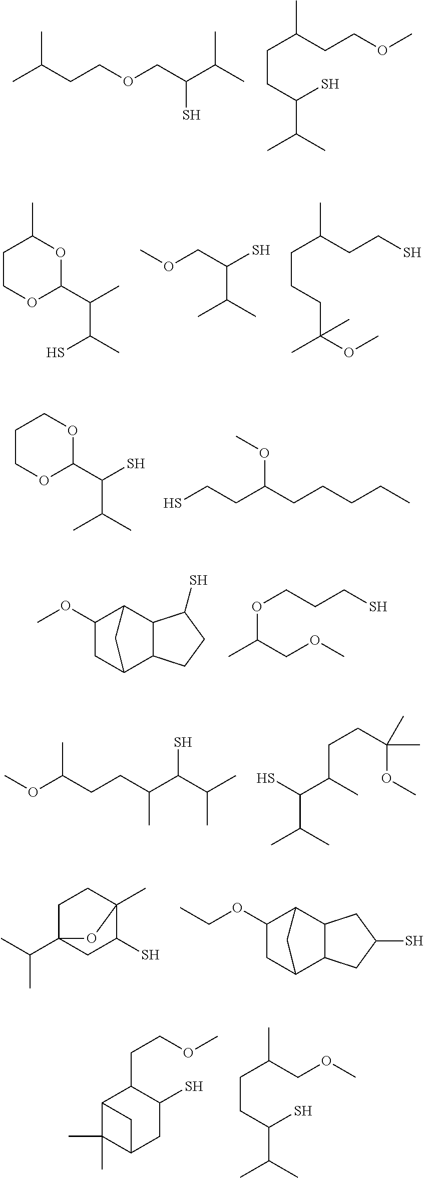

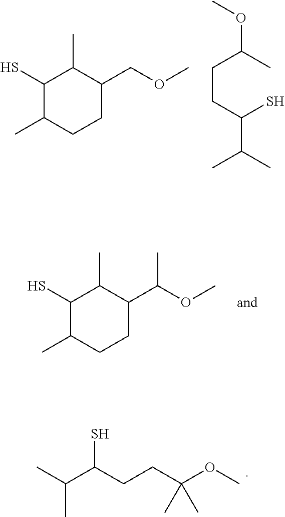

30. The compound of claim 16, wherein R¹, R² and R³ are independently selected from the groups as defined in (i), respectively, and R and R¹ are bonded together to form a saturated or unsaturated ring system comprising 5 to 10 carbon atoms, preferably wherein the ring system additionally carries one, two or more C₁ to C₅ linear, branched or cyclic alkyl, alkenyl or alkadienyl substituents,

31. The compound of claim 16, wherein R¹, R¹ and R² are independently selected from the groups as defined in (i), respectively, and R and R³ are bonded together to form a saturated or unsaturated, mono or bicyclic, ring system comprising 5 to 10 carbon atoms and optionally 1 or 2 ether functional groups, preferably wherein the ring system additionally carries one, two or more C₁ to C₅ linear, branched or cyclic alkyl, alkenyl or alkadienyl substituents.
